(19) United States
(12) Patent Application Publication
(10) Pub. No.: US 2021/0196851 A1
(43) Pub. Date: Jul. 1, 2021

(54) AIR TREATMENT SYSTEM EMPLOYING ULTRAVIOLET LIGHT EMITTERS

(71) Applicant: Samir A. Khan, Naperville, IL (US)

(72) Inventor: Samir A. Khan, Naperville, IL (US)

(21) Appl. No.: 17/192,880

(22) Filed: Mar. 4, 2021

(51) Int. Cl. A61L 9/20

(52) U.S. Cl. CPC A61L 9/20; A61L 2209/11; A61L 2209/111; A61L 2209/14

(57) ABSTRACT

An air treatment system includes a pathogen treatment chamber that is configured with one or more channel assemblies such that air passing therethrough is treated with ultraviolet (UV) light. A channel assembly includes multiple parallel channels configured between the opposed ends of the channel assembly. One end of each channel is configured with an ultraviolet (UV) light source exhibiting a first wavelength, while the remaining end of each channel is configured with an ultraviolet (UV) light source exhibiting a second wavelength. A removable light source module including the UV light sources exhibiting the first wavelength spans one of the opposed ends of the channels, while another removable UV light source module including the UV light sources exhibiting the second wavelength spans the remaining opposed ends of the channels.

### BACKGROUND

The disclosures herein relate generally to the treatment of pathogens, and more particularly, to the treatment of pathogens in air by exposing the air to ultraviolet light.

### BRIEF DESCRIPTION

In one embodiment, an air treatment apparatus is disclosed that includes a housing having a main air input and a main air output. The apparatus also includes at least one air mover situated within the housing to move air in an air flow path from the main air input to the main air output. At least one filter is situated in the air flow path. The apparatus includes a pathogen treatment chamber (PTC) situated within the housing and intersecting the air flow path. In more detail, the PTC includes a first channel assembly of parallel channels that are situated side-by-side one another, each channel including first and second opposed ends. The PTC also includes a first removable ultraviolet (UV) light source module situated spanning the first opposed ends of the first channel assembly of parallel channels and including a respective ultraviolet emitter exhibiting a first wavelength adjacent each first opposed end of the first channel assembly. The PTC further includes a second removable ultraviolet (UV) light source module situated spanning the second opposed ends of the first channel assembly of parallel channels and including a respective ultraviolet emitter exhibiting a second wavelength adjacent each second opposed end of the first channel assembly. The first channel assembly includes an air input opening situated at one outermost channel of the first channel assembly. The first channel assembly further includes an air output opening situated at an opposed outermost channel of the first channel assembly. The first channel assembly of parallel channels includes wall members with openings at respective alternating opposed ends of the first channel assembly to form a serpentine path through the first channel assembly from the air input opening of the first channel assembly to the air output opening of the first channel assembly.

### BRIEF DESCRIPTION OF THE DRAWINGS

The appended drawings illustrate only exemplary embodiments of the invention and therefore to not limit its scope because the inventive concepts lend themselves to other equally effective embodiments.

FIG. 1A is a side perspective view of one embodiment of the disclosed air treatment system.

FIG. 1B is an exploded side plan view of one embodiment of the disclosed air treatment system.

FIG. 1C is another side perspective view of the embodiment of the disclosed air treatment system of FIG. 1A.

FIG. 1D is another side perspective view of the embodiment of the disclosed air treatment system of FIG. 1A.

FIG. 1E is a side perspective view of one channel assembly of the pathogen treatment chamber (PTC).

FIG. 1F is a side perspective exterior view of a removable ultraviolet (UV) light source module of the channel assembly of FIG. 1E.

FIG. 1G is a side perspective interior view of the removable ultraviolet (UV) light source module of FIG. 1F.

FIG. 1H is a side plan view of the removable ultraviolet (UV) light source module of FIG. 1F.

FIG. 1I is a perspective view showing the interior of a removable ultraviolet (UV) light source module.

FIG. 1J is a perspective view showing the exterior of the removable ultraviolet (UV) light source module of FIG. 11.

FIG. 1K is a perspective view of wall retainer member that the pathogen treatment chamber (PTC) may employ.

FIG. 1L is a perspective view showing two channel assemblies in a stacked configuration that the pathogen treatment chamber (PTC) may employ ,

FIG. 1M is a cross-sectional view of a representative channel assembly.

FIG. 1N is a side perspective view of a channel assembly from which a sectional view is taken.

FIG. 1O is a cross-sectional view of another representative channel assembly.

FIG. 1P is a perspective view of a top interposer component.

FIG. 1Q is a perspective view of a bottom interposer component.

FIG. 1R is a perspective view of the top interposer component mated with the bottom interposer component to form a complete interposer.

FIG. 1S is a perspective view of a first channel assembly with a bottom interposer component thereon.

FIG. 1T is a perspective view of a second channel assembly with a top interposer component thereon.

FIG. 1U is a perspective view of the channel assembly of FIG. 1S mated with the channel assembly of FIG. 1T via an interposer therebetween.

FIG. 2A is a side perspective view of another embodiment of the disclosed air treatment system.

FIG. 2B is a side plan view of the embodiment of the disclosed air treatment system of FIG. 2A.

FIG. 2C is a perspective view of a connecting pipe joining two channel assemblies of the air treatment system of FIG. 2A.

FIG. 2D is side plan view of a channel assembly of the air treatment system of FIG. 2A including a section line 2E-2E.

FIG. 2E is a sectional view of the channel assembly of FIG. 2D taken along section line 2E-2E.

FIG. 3A is a perspective view of another embodiment of the disclosed air treatment system.

FIG. 3B is another perspective view of the disclosed air treatment system of FIG. 3A.

FIG. 3C is a bottom perspective view of the disclosed air treatment system of FIG. 3A.

FIG. 3D is a top plan view of the disclosed air treatment system of FIG. 3A.

FIG. 3E is a side plan view of two representative channel assemblies of the disclosed air treatment system of FIG. 3A.

FIG. 3F is a cross sectional view of the two representative channel assemblies of the disclosed air treatment system of FIG. 3E.

FIG. 4A is a top perspective view of yet another embodiment of the disclosed air treatment system.

FIG. 4B is another top perspective view of the disclosed air treatment system of FIG. 4A.

FIG. 4C is a bottom side perspective view of disclosed air treatment system of FIG. 4A.

FIG. 4D is a top plan view of the disclosed air treatment system of FIG. 4A.

FIG. 4E is a side plan view of the disclosed air treatment system of FIG. 4A showing section line 4F-4F.

FIG. 4F is a cross sectional view of the disclosed air treatment system of FIG. 4E taken along section line 4F-4F.

FIG. 5A is a perspective view of a removable UV light source module including a UV LED strip installed therein.

FIG. 5B is a plan view of the removable UV light source module of FIG. 5A.

FIG. 5C is a representation of three removable UV light source modules coupled to one another for power distribution purposes.

FIG. 6A is a perspective view of one embodiment of the air treatment system including a frame.

FIG. 6B is a perspective view of the air treatment system of FIG. 6A shown with a housing covering the frame.

FIG. 6C is a perspective view of the air treatment system of FIG. 6A shown with a system controller installed on the lower funnel thereof.

FIG. 6D is a perspective view of the air treatment system wherein the housing includes an upper housing section, a mid-housing section and a lower housing section that provides door access to the filters of the system.

FIG. 7 is high level block diagram of the system controller of the air treatment system shown together with electrical connections to UV sensors, air filter sensors and an air mover sensor.

FIG. 8A shows the channel assembly of FIG. 4F illustrated with screw receivers to demonstrate one way to removably connect a UV light source module to the wall structure of a channel assembly.

FIG. 8B shows a plan view of one end of a channel assembly of FIG. 8A showing a simplified first arrangement of screws to removably connect a UV light source module to the wall structure of the channel assembly.

FIG. 8C shows a plan view of one end of a channel assembly of FIG. 8A showing a more detailed second arrangement of screws to removably connect a UV light source module to the wall structure of the channel assembly.

FIG. 9 shows a perspective view of a channel assembly wherein two removable UV light source modules are coupled to a wall structure via toggle latch fasteners.

### DETAILED DESCRIPTION

Over the years, pathogens such as bacteria, viruses, fungi and parasites have continued their attack on humanity. Of these pathogens, bacteria and viruses can be airborne and thus especially infectious even without direct contact between humans. For example, in 2020 and continuing into 2021, the airborne SARS-CoV-2virus spread throughout the world from individual to individual without direct contact. An air treatment system to treat air containing such viruses to inactivate, neutralize disable or otherwise ameliorate the viruses is very desirable. One goal of the disclosed air treatment system is to decontaminate pathogen-laden air to make the air again breathable by humans. The disclosed air treatment system can provide a form of air purification for air containing undesired pathogens.

### Component List

The following component list is provided as a convenience to the reader. The disclosed technology is not limited to only these components that are recited below for purposes of example.

100 air treatment system

101 main air input

102 main air output

103 grill

120 pathogen treatment chamber (PTC)

120A PTC air input

120B PTC air output

121 channel assembly

122 channel assembly

123 channel assembly

124 channel assembly

125 channel assembly

126 channel assembly

121A channel of channel assembly 121

121B channel of channel assembly 121

121C channel of channel assembly 121

121D channel of channel assembly 121

121E channel of channel assembly 121

121F channel of channel assembly 121

121-FE front end

121-RE rear end

121-S1 opposed side

121-S2 opposed side

121-1 UV light source

121-2 UV light source

121-3 UV light source

121-4 UV light source

121-5 UV light source

121-6 UV light source

121-1′ UV light source

121-2′ UV light source

121-3′ UV light source

121-4′ UV light source

121-5′ UV light source

121-6′ UV light source

121-IN air input opening

121-OUT air output opening

121-WS wall structure

121-WS-R wall retainer member—e.g. rectangular, parallelepiped

121-W1 wall

121-W2 wall

121-W3 wall

121-W4 wall

121-W5 wall

121-W1H wall hole/opening

121-W2H wall hole/opening

121-W3H wall hole/opening

121-W4H wall hole/opening

121-W5H wall hole/opening

121-WS-A closed side of wall retainer 121-WS-R

121-WS-B closed side of wall retainer 121-WS-R

121-WS-C closed side of wall retainer 121-WS-R

121-WS-D closed side of wall retainer 121-WS-R

121-WS-E open portion of sidewall structure 121-WS

121-WS-F open portion of sidewall stricture 121-WS

122-IN air input opening

122-OUT air output opening

122-WS wall structure

122-WS-R wall retainer member—e.g. rectangular, parallelepiped

122-W1 wall

122-W2 wall

122-W3 wall

122-W4 wall

122-W5 wall

122-W1H wall hole/opening

122-W2H wall hole/opening

122-W3H wall hole/opening

122-W4H wall hole/opening

122-W5H wall hole/opening

122-WS-A closed side of wall retainer 122-WS-R

122-WS-B closed side of wall retainer 122-WS-R

122-WS-C closed side of wall retainer 122-WS-R

122-WS-D closed side of wall retainer 122-WS-R

122-WS-E open portion of sidewall structure 122-WS

122-WS-F open portion of sidewall structure 122-WS

122-1, 122-2, 122-3, 122-4, 122-5 and 122-6 UV light sources

122-1′, 122-2′, 122-3′, 122-4′, 122-5′ and 122-6′ UV light sources

127 funnel

127A funnel input,

127B funnel output

130 funnel

130A funnel input,

130B funnel output

131 filter

132 filter

133 filter

135 air moving device (air mover)

140-1 ultraviolet (UV) light source module

140-1E end surface of UV light source module 140-1

140-1R rear end of UV light source module

140-2 UV light source module

140-2R rear end of UV light source module

150-1 ultraviolet (UV) light source module

150-1R rear end of UV light source module

150-2 UV light source module

150-2R rear end of UV light source module

160 top interposer component

160A female interconnect

160B female interconnect

160C female interconnect

160D female interconnect

161 bottom interposer component

161A male interconnect

161B male interconnect

161C male interconnect

161D male interconnect

162 top interposer component

162A female interconnect

162B female interconnect

162C female interconnect

162D female interconnect

163 bottom interposer component

163A female interconnect

163B female interconnect

170 2-piece mating interposer assembly

181 gasket

182 gasket

220 PTC

220A input of PTC

220B output of PTC

221 channel assembly

222 channel assembly

223 channel assembly

224 channel assembly

225 channel assembly

226 channel assembly

221-FE front end

221-RE rear end

221-S1 opposed side

221-S2 opposed side

221-1 UV light source

221-2 UV light source

221-3 UV light source

221-4 UV light source

221-5 UV light source

221-6 UV light source

221-A channel

221-B channel

221-C channel

221-D channel

221-E channel

221-F channel

221-W1 wall

222-W2 wall

222-W3 wall

221-W4 wall

221-W5 wall

221-IN input of channel assembly 221

221-OUT input of channel assembly 221

222-IN input of channel assembly 222

222-OUT input of channel assembly 222

223 channel assembly

223-IN input of channel assembly 223

223-OUT input of channel assembly 223

224 channel assembly

224-IN input of channel assembly 224

224-OUT input of channel assembly 224

225 channel assembly

225-IN input of channel assembly 225

225-OUT input of channel assembly 225

226 channel assembly

226-IN input of channel assembly 226

226-OUT input of channel assembly 226

240-1 ultraviolet (UV) light source module

250-1 ultraviolet (UV) light source module

251 connecting pipe (channel assembly connector)

252 connecting pipe(channel assembly connector)

253 connecting pipe (channel assembly connector)

254 connecting pipe (channel assembly connector)

255 connecting pipe (channel assembly connector)

256 connecting pipe (channel assembly connector)

300 air treatment system

321 channel assembly

321-IN input of channel assembly 321

321-OUT output of channel assembly 321

322 channel assembly

322-IN channel assembly input

322-OUT channel assembly output

323 channel assembly

323-IN channel assembly input

323-OUT channel assembly output

324 channel assembly

324-IN channel assembly input

325 channel assembly

322-FE front end

322-RE rear end

322-S1 opposed side

322-S2 opposed side

322-A channel

322-B channel

322-C channel

322-D channel

322-E channel

322-F channel

322-G channel

322-W1 wall

322-W2 wall

322-W3 wall

322-W4 wall

322-W5 wall

322-W6 wall

323-A channel

323-B channel

323-C channel

323-D channel

323-E channel

322-F channel

322-G channel

340-2 ultraviolet (UV) light source module

350-2 ultraviolet (UV) light source module

351 connecting pipe

352 connecting pipe

353 connecting pipe

354 connecting pipe

355 connecting pipe

356 connecting pipe

400 air treatment system

421 channel assembly

422 channel assembly

422-IN channel assembly input

422-OUT channel assembly output

423 channel assembly

423-IN channel assembly input

423-OUT channel assembly output

424 channel assembly

424-IN channel assembly

425 channel assembly

426 channel assembly

422-FE front end

422-RE rear end

422-S1 opposed side

422-S2 opposed side

422-A channel

422-B channel

422-C channel

422-D channel

422-E channel

422-F channel

422-G channel

422-WS wall structure (includes wall-retainer 422-WS-R)

422-WS-R wall retainer

422-W1 wall

422-W2 wall

422-W3 wall

422-W4 wall

422-W5 wall

422-W6 wall

422-1 UV light source

422-2 UV light source

422-3 UV light source

422-4 UV light source

422-5 UV light source

422-6 UV light source

423-A channel

423-B channel

423-C channel

423-D channel

423-E channel

422-F channel

422-G channel

440-2 UV light source module

450-2 UV light source module

452 connector valve (channel assembly connector)

453 connector valve (channel assembly connector)

454 connector valve (channel assembly connector)

455 connector valve (channel assembly connector)

456 connector valve (channel assembly connector)

457 connector valve (channel assembly connector)

502 UV LED strip

504 mounting screw

506 mounting screw

508 mounting screw

510 positive power rail

510A end terminal

510B end terminal

512 negative power rail

512A end terminal

512B end terminal

601 frame member

602 frame member

603 frame member

604 frame member

610 housing enclosure

610-1 sub-housing

610-2 sub-housing

610-3 sub-housing

612 top of housing

614 bottom of housing

620 door

625 latch

630 hinge

700 system controller

705 processor

710 control device

715 Bluetooth transceiver

720 power controller

722 manual control

724 power connector

725 UV sensors

730 air filter sensors

735 air mover sensor

801 screw receiver

802 hole in screw receiver

803 screw

811 screw receiver

812 hole in screw receiver

813 screw

822 UV light strip

900 channel assembly

905 UV light source module

905-1 UV light source

905-2 UV light source

905-3 UV light source

905-4 UV light source

905-5 UV light source

905-6 UV light source

910 UV light source module

910-IN air input

915-WS wall structure

915-WS-R wall retainer of wall structure 915-WS.

915-WS-A wall structure side

915-WS-B wall structure side

915-WS-C wall structure side

915-WS-D wall structure side

921 toggle latch

922 toggle latch

923 toggle latch

924 toggle latch

981 silicone gasket

982 silicone gasket

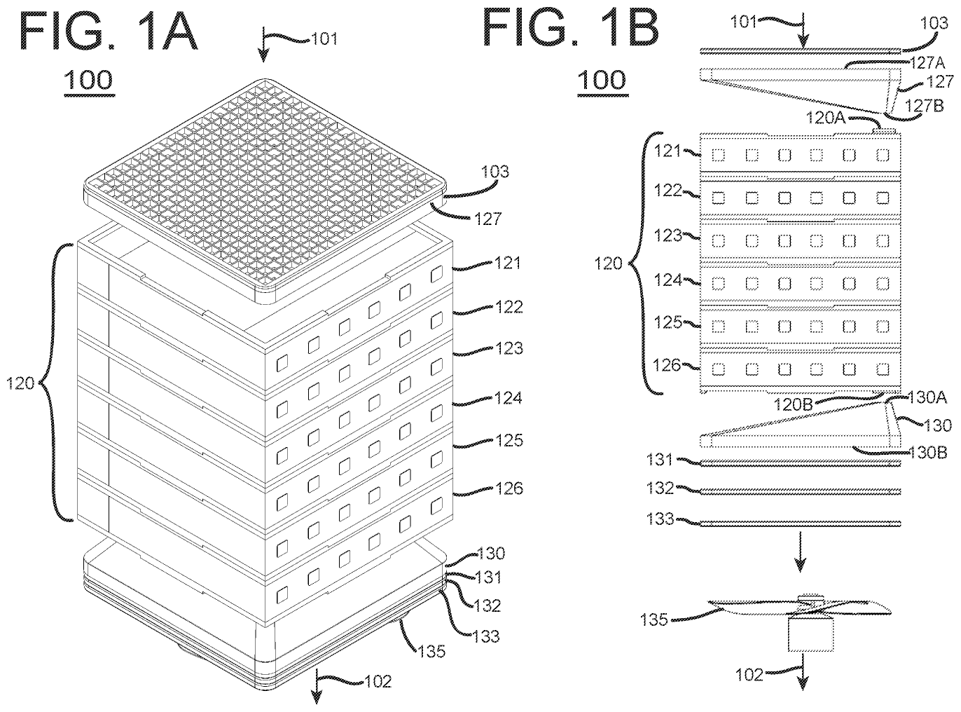

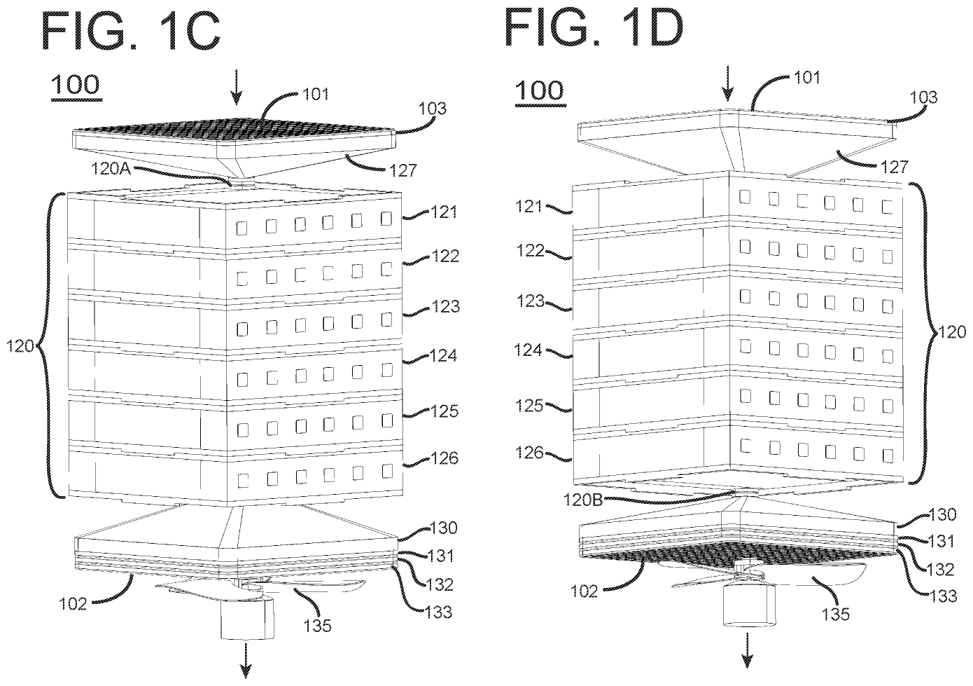

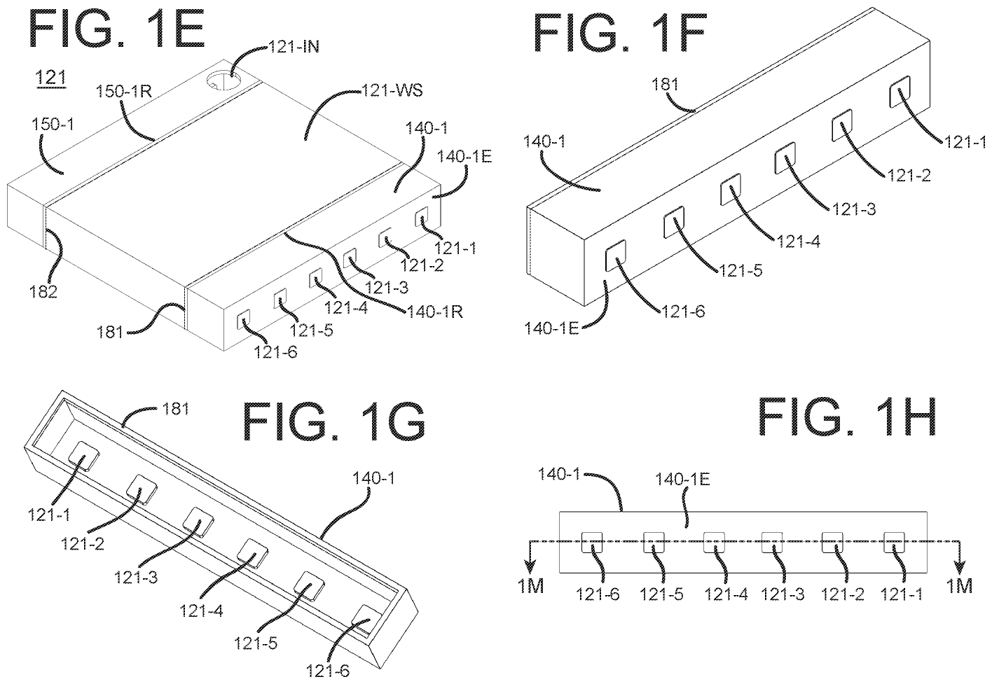

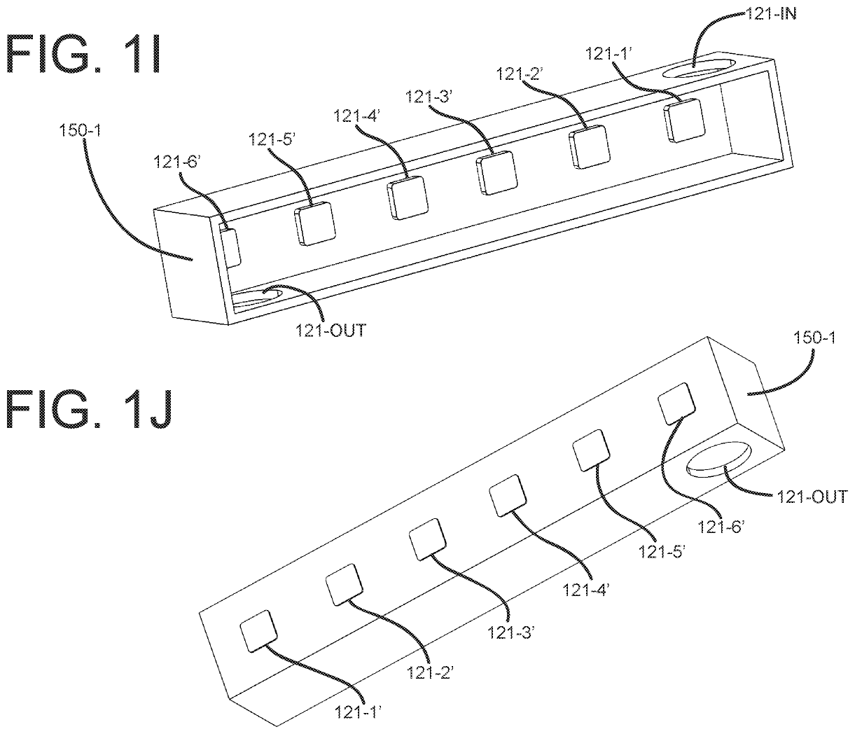

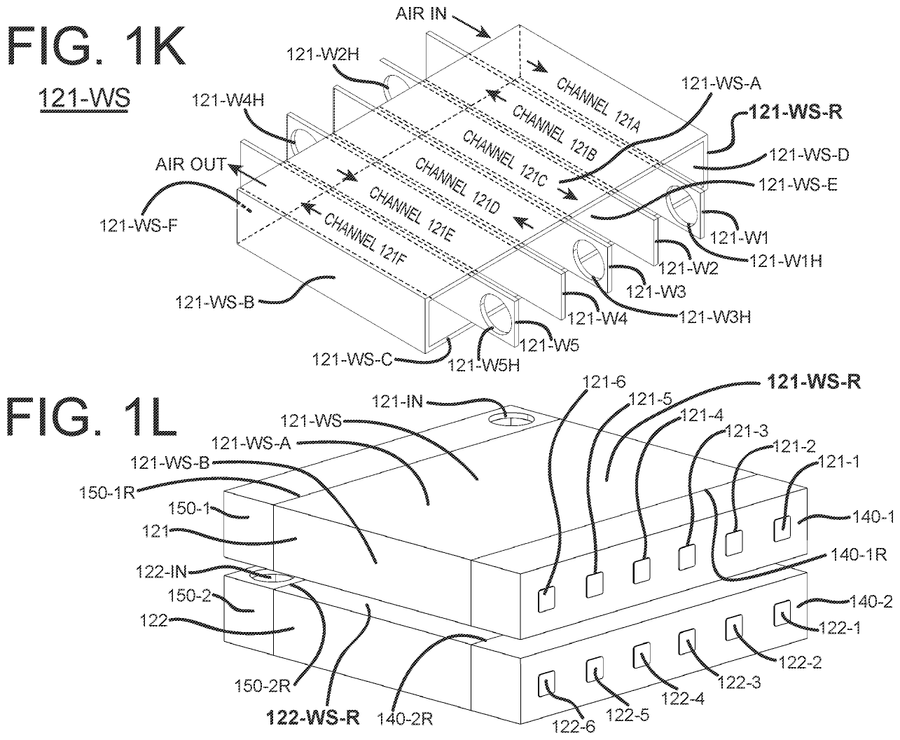

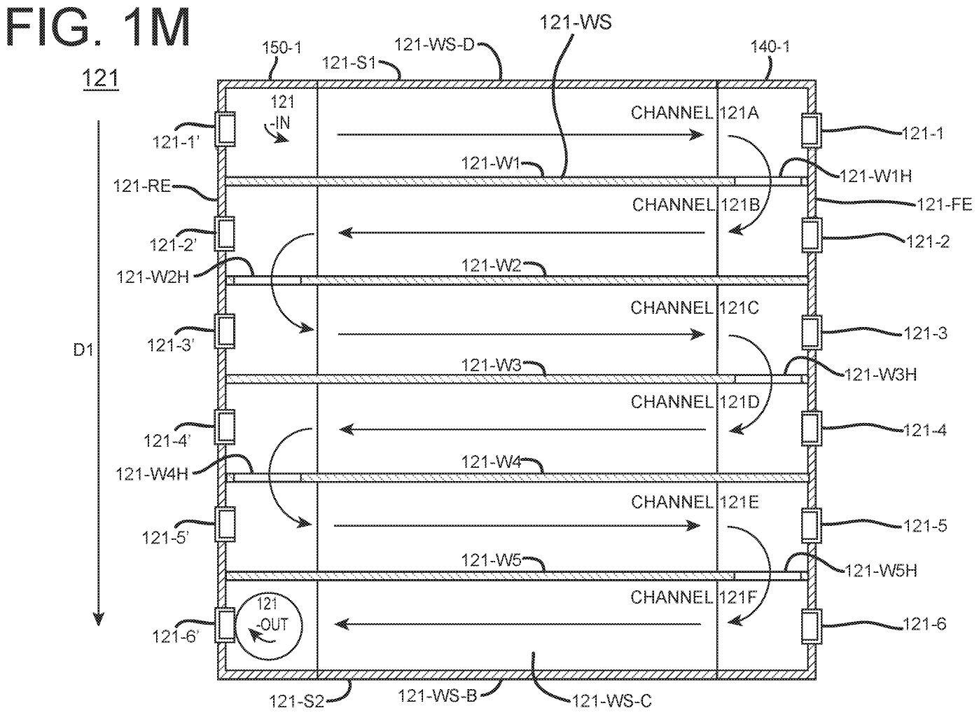

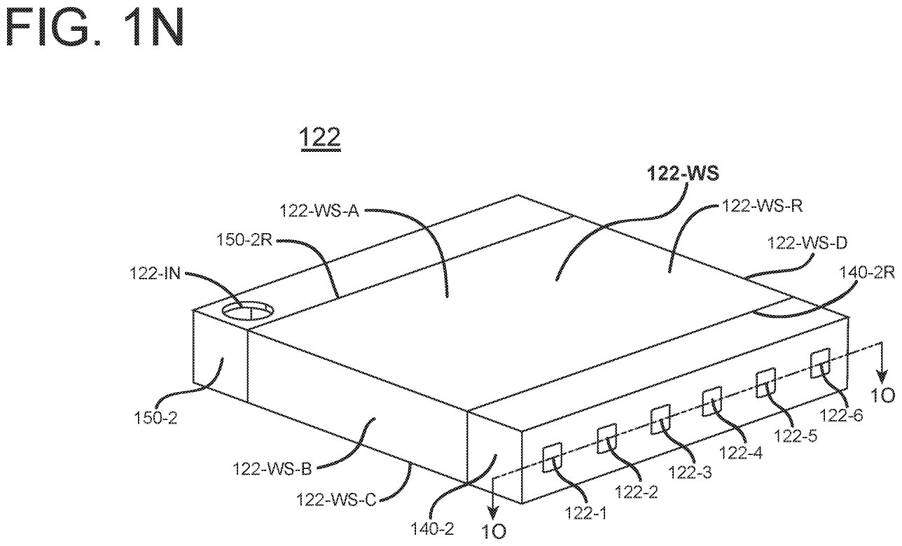

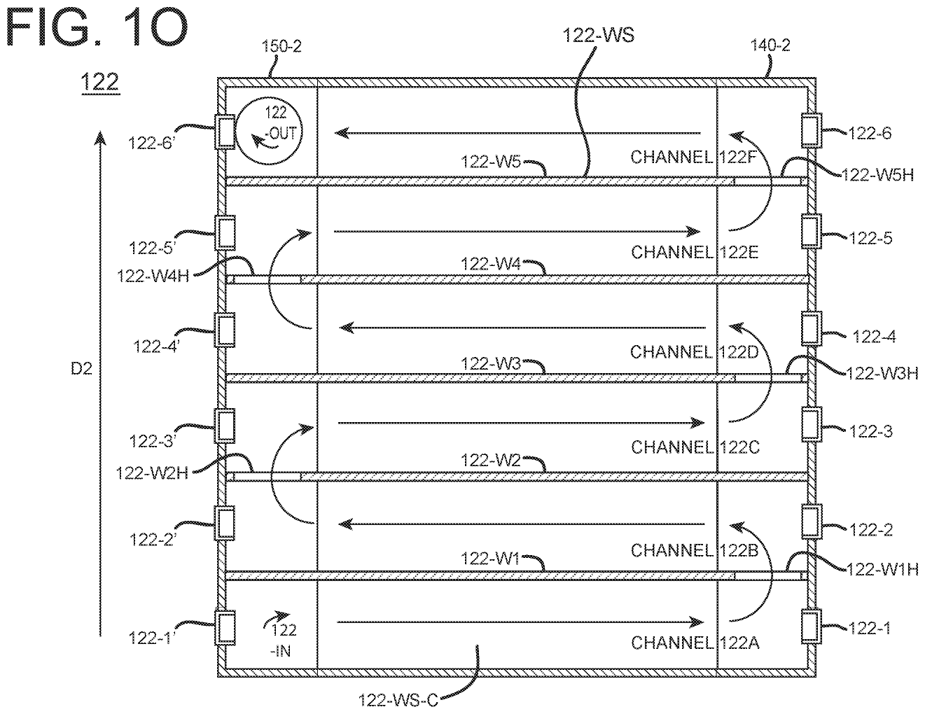

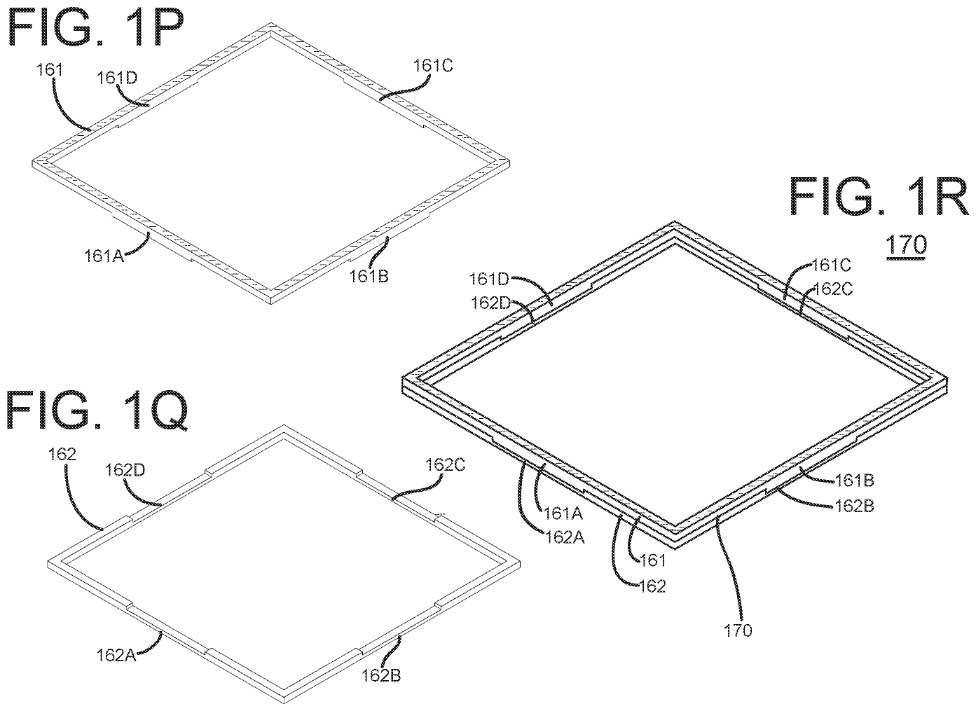

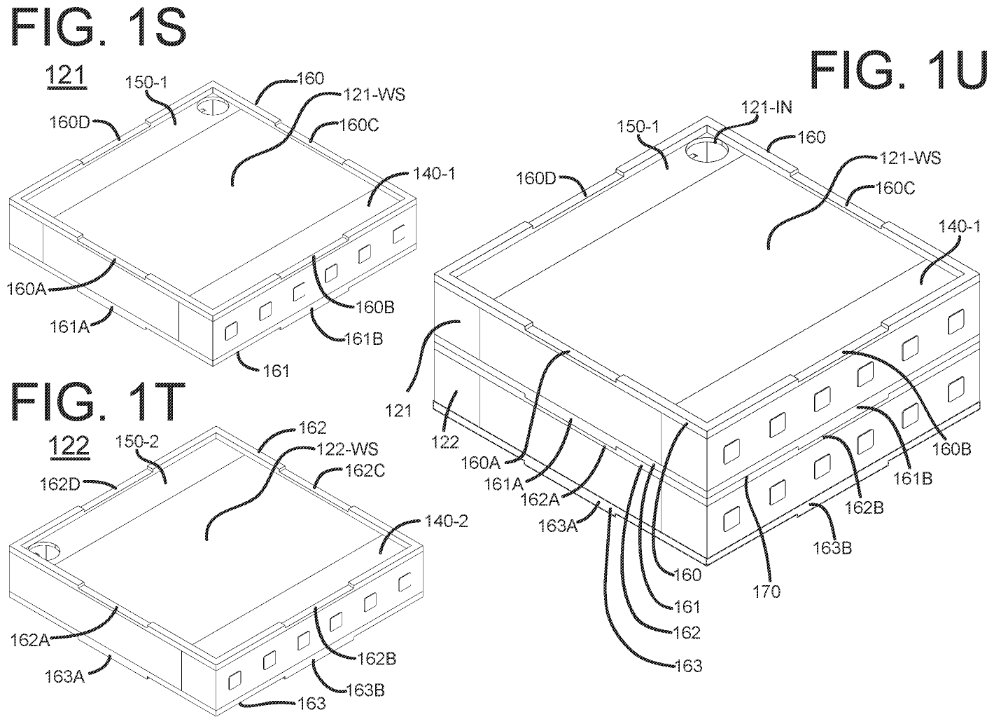

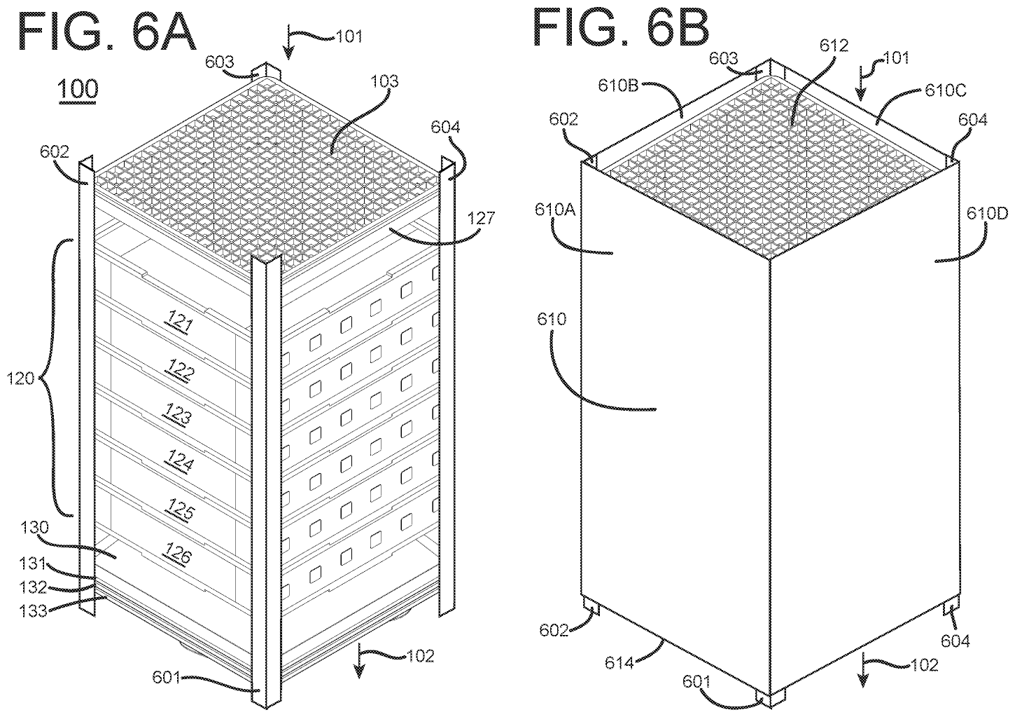

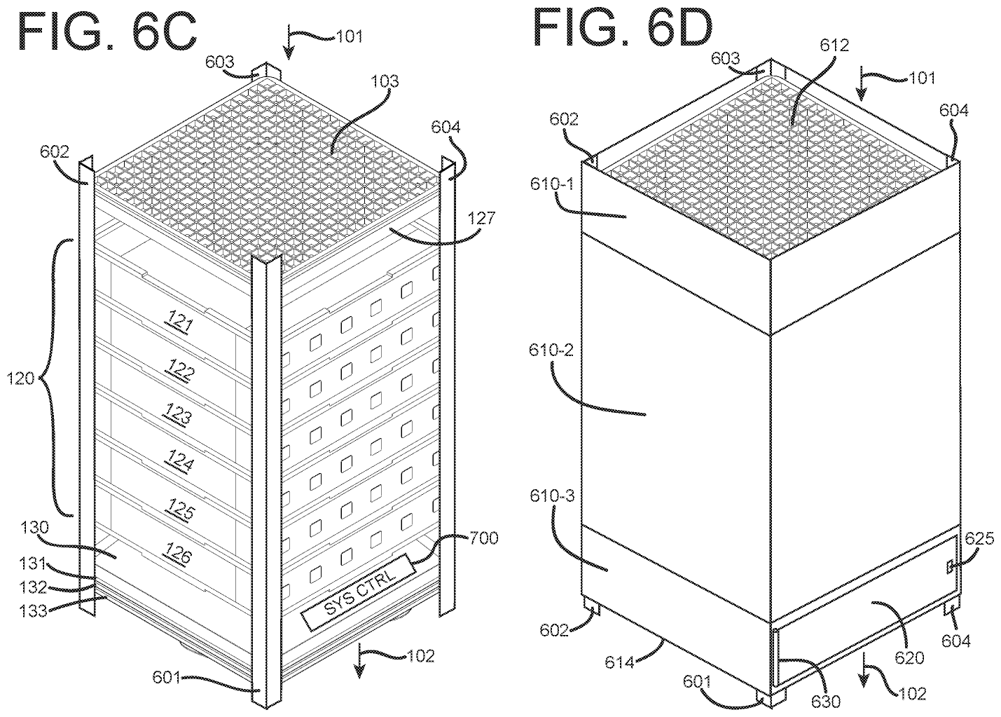

FIG. 1A shows a side perspective view of one embodiment of the disclosed air treatment system as air treatment system 100, while FIG. 1B shows an exploded side plan view of air treatment system 100. The air flow path through system 100 is depicted generally using downward facing arrows in FIGS. 1A and 1B. System 100 includes a main air input 101 and a main air output 102. In actual practice, system 100 may include a housing that substantially surrounds all or a portion of the system depicted in FIG. 1A. A representative housing 610 is depicted in FIGS. 6A-6D. Returning to FIG. A grill 103 is situated at main air input 101 and includes grill openings shaped in a grid pattern sufficiently small to prevent large particles from entering the air flow path of system 100.

Describing now at a high level, in one embodiment system 100 includes a pathogen treatment chamber (PTC) 120 containing 6 channel assemblies 121, 122, 123, 124, 125 and 126 in this particular example. More or fewer channel assemblies may be employed by system 100 depending on the desired application, and the rate of, and the amount of, treatment desired. In particular, more or fewer channel assemblies may be employed by PTC 120 of system 100 depending on the size (i.e. volume) of the particular usage environment and the desired air flow rate measured as cubic feet per minute (CFM) based on cooling or heat capacity per ton, to ensure sufficient exposure of the pathogen in the air flow path (i.e. sufficient dosage) to achieve the desired amount of air treatment. In one embodiment, system 100 includes at least one channel assembly 121. In another embodiment, system 100 includes at least two channel assemblies such as channel assemblies 121 and 122. The channel assemblies are modular and may be stacked one upon the other as shown in FIG. 1A. As seen in FIG. 1B, a funnel 127 is situated between grill 103 and an air input 120A of PTC 120. Funnel 127 includes wide and narrow ends that form a wide funnel input 127A at one end of the funnel 127, and form a narrower funnel output 127B at the opposite end of the funnel 127. Funnel input 127A is situated below grill 103 to receive untreated air passing through grill 103. Funnel 127 concentrates air received from funnel input 127A and provides that air to funnel output 127B that communicates the air passing therethrough to the PTC 120 that is situated below funnel 127. More specifically, funnel output 127B communicates air from grill 103 to the air input 120A of PTC 120.

System 100 further includes a funnel 130 situated between air output 120B of PTC 120 and filters 131, 132 and 133. Funnel 130 includes a narrow end and a wide end that form a funnel input 130A and funnel output 130B, respectively. PTC output 120B communicates the air treated by PTC 120 to filters 131, 132 and 133 that are situated below funnel output 130B, as shown in FIG. 1B. Filters 131, 132 and 133 provide additional cleaning of the air treated by PTC 120. By way of example and not limitation, in one embodiment, filter 131 may be an Ultra-Low Particulate Air (ULPA) filter with a MERV rating of at least approximately 17.) In one embodiment, filter 132 may be an Ultra-Low Particulate Air (ULPA) filter with a MERV rating of at least approximately 13.) In one embodiment, filter 133 may be an activated carbon filter. An air moving device 135, i.e. an air mover, is situated below filters 131, 132, and 133 to pull air from main air input 101 of system 100 through to main air output 102 of system 100 as shown in FIG. 1A and FIG. 1B. In one embodiment, air moving device 135 is a fan, while in another embodiment air moving device 135 is an impeller that sucks air from main air input 101 through grill 103, PTC 120, filters 131-133 and any intervening structures in the air flow path between main air input 101 and main air output 102. While air mover 135 may be a propeller or impeller, an impeller is preferred. The terms “downstream” and “upstream” may be used to describe some of the components of system 100 in terms of the location of such components relative to one another within the air flow path from main air input 101 to main air output 102. For example, filter 131 is downstream in the air flow path relative to funnel 130 of FIG. 1B. Similarly, channel assembly 121 is upstream of filter 131 of FIG. 1B.

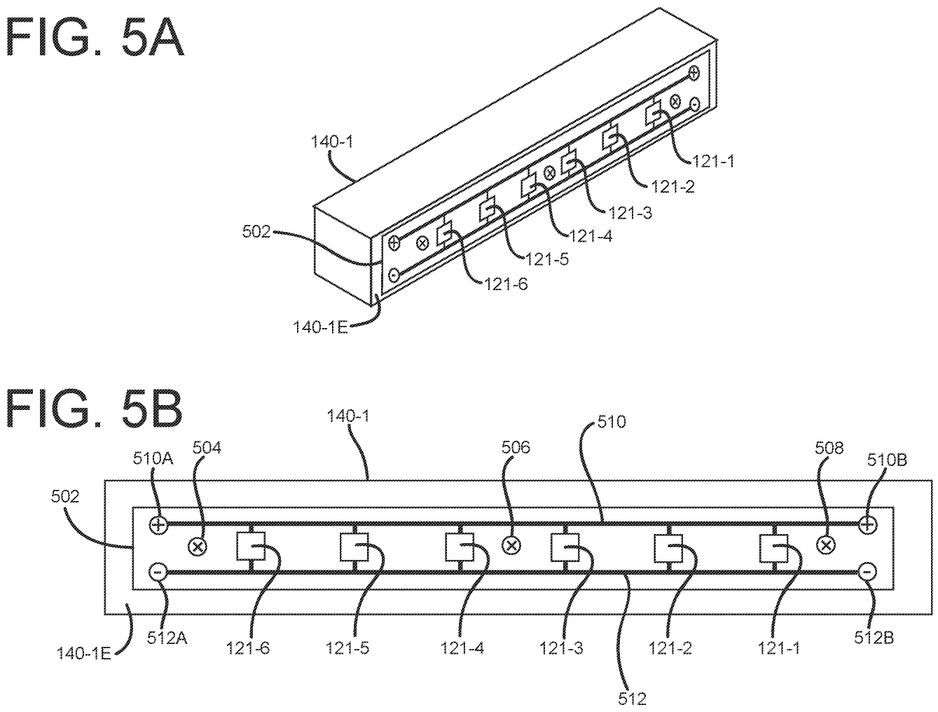

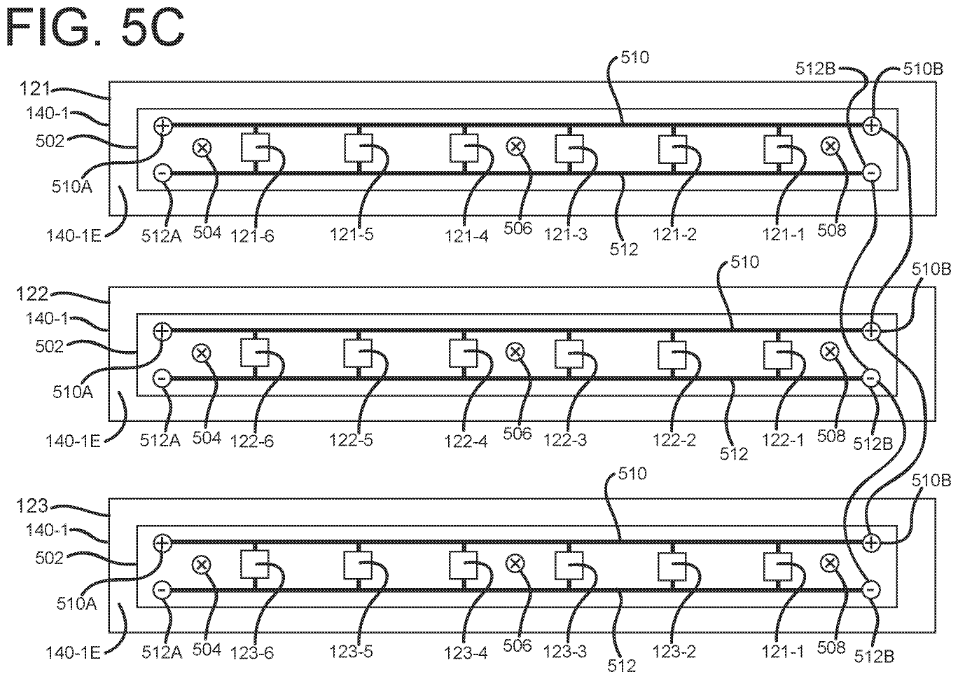

A representative channel assembly 121 of channel assemblies 121-126 is now discussed by way of example. These teachings with respect to channel assembly 121 can be generally applied to each of the channel assemblies taken together with the noted differences. Whereas FIG. 1C and FIG. 1D show channel assembly 121 “in situ” within PTC 120, FIG. 1E shows channel assembly 121 only. Referring now to FIG. 1K, a wall structure 121-WS is shown before channel assembly 121 is assembled. Channel assembly 121 includes this wall structure 121-WS which exhibits a plurality of parallel channels 121A, 121B, 121C, 121D, 121E and 121F situated therein, as described in more detail below with reference to FIG. 1M. These parallel channels are internal to channel assembly 121 and are thus not visible in FIG. 1E. Continuing now with FIG. 1E, channel assembly 121 includes removable ultraviolet (UV) light source module 140-1 that includes ultraviolet (UV) light sources 121-1, 121-2, 121-3, 121-4, 121-5 and 121-6 which are removably mounted thereon using screws 504, 506 and 508 as seen in FIG. 5B. As discussed in more detail with reference to FIGS. 5A and 5B, a UV LED strip 502 that includes UV LEDs 121-1, 121-2, 121-3, 121-4, 121-5 and 121-6 is mounted to UV light source module 140-1 by screws 504, 506 and 508 that hold UV LED strip 502 to front end 140-1E. This attachment arrangement facilitates removal of UV LED strip 502 from wall structure 121-WS to permit replacement of UV LED strip 502 when one or more of the UV LEDs thereon fails.

Turning now again to FIG. 1E, UV light source module 121 includes an air-tight gasket 181 situated between UV light source module 140-1 and wall structure 121-WS. UV light source module 121 also includes an air-tight gasket 182 situated between UV light source module 150-1 and wall structure 121-WS. In this manner, gaskets 181 and 182 seal channel assembly 121 such that the air provided to air input opening 121-IN of FIG. 1E travels through channel assembly 121 and reaches air output opening 121-OUT of FIGS. 11 and 1J without leaking from channel assembly 121 while flowing therethrough. For simplicity, FIGS. 11 and 1J are drawn without gasket 182 thereon. Gasket 182 is visible in FIG. 1E. Gaskets 181 and 182 are preferably silicone gaskets, while other flexible rubber-like materials may be employed as well as air-tight seals that prevent air leakage along the air flow path of a channel assembly.

Turning once again to FIG. 1F, (UV) light sources 121-1, 121-2, 121-3, 121-4, 121-5 and 121-6 are mounted in UV light source module 140-1 as viewed externally to module 140-1. FIG. 1G shows the same UV light sources 121-1, 121-2, 121-3, 121-4, 121-5 and 121-6 mounted in UV light source module 140-1 as viewed internally. FIG. 1H shows a plan view of end surface 140-1E of module 140-1 together with a section line 1M-1M.

FIG. 11 shows UV light sources 121-1′, 121-2′, 121-3′, 121-4′, 121-5′ and 121-6′ mounted in UV light source module 150-1 as viewed internally. UV light source module 150-1 includes an air input opening 121-IN and an air output opening 121-OUT. FIG. 11 shows the same UV light sources 121-1′, 121-2′, 121-3′, 121-4′, 121-5′ and 121-6′ mounted in UV light source module 150-1 as viewed externally.

FIG. 1K shows the wall structure 121-WS that forms the channels of channel assembly 121. More particularly, wall structure 121-WS includes a wall retainer member 121-WS-R that in one embodiment exhibits a rectangular parallelepiped geometry with four (4) closed sides and two (2) open ends, as illustrated. The four (4) closed sides 121-WS-A, 121-WS-B, 121-WS-C and 121-WS-D hold the spaced-apart parallel walls 121-W1, 121-W2, 121-W3, 121-W4 and 121-W5 in position to form channels 121A, 121B, 121C, 121D, 121E and 121F as shown. Side wall 121-WS-B and side wall 121-WS-D form part of channel 121F and channel 121A, respectively. It is noted that walls 121-W1, 121-W2, 121-W3, 121-W4 and 121-W5 extend beyond both open ends 121-WS-E and 121-WS-F of wall structure 121-WS. Wall structure 121-WS including both wall retainer 121-WS-R and walls 121-W1, 121-W2, . . . 121-W5 may be molded from PVC or other moldable material such that the components thereof are integral one another to form a single unitary structure. In other words, wall structure 121-WS may be a unitary structure that includes wall retainer member 121-WS-R and walls 121-W1, 121-W2, 121-W3, 121-W4 and 121-W5 therein. Walls 121-W1, 121-W2, 121-W3, 121-W4 and 121-W5 respectively include wall holes, i.e. openings, 121-W1H, 121-W2H, 121-W3H, 121-W4H and 121-W5H, at alternating ends thereof to allow air to flow from one channel to an adjacent channel through these openings.

FIG. 1L shows two of the channel assemblies of FIGS. 1A and 1B in a stacked configuration, namely channel assembly 121 is situated atop channel assembly 122 below. Treated air from channel assembly 121 flows into channel assembly 122 for additional treatment. Additional channel assemblies can be stacked below channel assemblies 121 and 122 for increased air treatment if desired. The disclosed air treatment system is thus modular and customizable according to the particular user's needs. Adding more channel assemblies in this manner increases the time during which the pathogen is exposed to UV light while being treated by PTC 120. This allows PTC 120 to more completely treat the air passing therethrough to better neutralize the pathogen. Preferably, system 100 treats the air passing therethrough for approximately 30 seconds or more.

To construct channel assembly 121, the builder positions removable UV light source module 140-1 over the ends of walls 121-W1, 121-W2, 121-W3, 121-W4 and 121-W5 such that the rear 140-1R of UV light source module 140-1 is flush with wall retainer member 121-WS-R, as shown in FIG. 1L. Likewise, the builder positions removable UV light source module 150-1 over the remaining ends of walls 121-W1, 121-W2, 121-W3, 121-W4 and 121-W5 such that the rear 150-1R of UV light source module 150-1 is flush with wall retainer member 121-WS-R, as illustrated. The builder need not necessarily be a human builder, but may be a robotic builder as well.

FIG. 1M is a cross-sectional view of a representative channel assembly 121 of FIG. 1H taken along section line 1M-1M. FIG. 1M shows the interior of channel assembly 121 as including walls 121-W1, 121-W2, 121-W3, 121-W4 and 121-W5 that define channels 121A, 121B, 121C, 121D, 121E and 121F. For clarity, front end 121-FE is the end of channel assembly 121 where UV light sources 121-1, 121-2, . . . 121-6 are located, whereas the opposite end of channel assembly 121 is considered to be the rear end 121-RE of channel assembly 121 where the remaining UV light sources 121-1′, 121-2′, . . . 121-6′ are located. In contrast, opposed sides 121-S1 and 121-S2 are the sides of channel assembly 121 running along the two outer opposed channels 121A and 121F, respectively.

The air flow path through representative channel assembly 121 is now described. Straight and curved arrows illustrate the air flow path from air input opening 121-IN to air output opening 121-OUT in channel assembly 121. Since air input opening 121-IN is above the section line 1M-1M, air input opening 121-IN is not actually visible in FIG. 1M. Nonetheless, air input opening 121-IN is marked in FIG. 1M to help inform the reader where air flow enters channel assembly 121 as seen more clearly in FIG. 1L.

In more detail with reference again to FIG. 1M, air to be treated by channel assembly 121 passes through air input opening 121-IN where adjacent UV light source 121-1′ treats the air with ultraviolet (UV) light. The treated air travels along channel 121A from the end of channel 121A adjacent UV light source 121-1″ to the opposite end of channel 121A where UV light source 121-1 further treats the treated air. The treated air flows through wall hole 121-W1H and into the end of channel 121B adjacent UV light source 121-2 which further treats the air with UV light. Now the treated air travels along channel 121B from the end of channel 121B adjacent UV light source 121-2 to the opposite end of channel 121B where UV light source 121-2′ further treats the treated air. The treated air then passes through wall hole 121-W2H and into the end of channel 121C adjacent UV light source 121-3′ which further treats the air with UV light.

The treated air travels along channel 121C from the end of channel 121C adjacent UV light source 121-3′ to the opposite end of channel 121C where UV light source 121-3 further treats the treated air. The treated air passes through wall hole 121-W3H and into the end of channel 121D adjacent UV light source 121-4 which further treats the air with UV light. Next, the treated air travels along channel 121D from the end of channel 121D adjacent UV light source 121-4 to the opposite end of channel 121D where UV light source 121-4′ further treats the treated air. The treated air passes through wall hole 121-W4H and into the end of channel 121E adjacent UV light source 121-5′ which further treats the air with UV light.

The treated air travels along channel 121E from the end of channel 121E adjacent UV light source 121-5′ to the opposite end of channel 121E where UV light source 121-5 further treats the treated air. The treated air passes through wall hole 121-W5H and into the end of channel 121F adjacent UV light source 121-6 which further treats the air with UV light. Now the treated air travels along channel 121F from the end of channel 121F adjacent UV light source 121-6 to the opposite end of channel 121F where UV light source 121-6′ further treats the treated air. The treated air exits channel 121F and channel assembly 121 at air output opening 121-OUT. In the embodiment of FIG. 1M, channel 121 and channel 121F are considered to be the two outermost channels.

As noted above with reference to FIG. 1K, FIG. 1L and FIG. 1M, walls 121-W1, 121-W2, 121-W3, 121-W4 and 121-W5, together with the closed sides 121-WS-A, 121-WS-B, 121WS-C and 121-WS-D of wall retainer 121-WS-R, form channels 121A, 121B, 121C, 121D, 121E and 121F. These walls may also be referred to as wall members. Wall members 121-W1, 121-W2, 121-W3, 121-W4 and 121-W5 include openings at respective alternating opposed ends of channel assembly 121 that form a serpentine air flow path through channel assembly 121 from air input opening 121-IN to air output opening 121-OUT. For example, as seen in FIG. 1M, wall member 121-W1 includes opening 121-W1H at one end of channel assembly 121 while adjacent wall 121-W2 includes opening 121-W2H at the alternating opposed end of channel assembly 121. Continuing further along the air flow path of channel assembly 121, wall 121-W3 which is adjacent to wall 121-W2 includes opening 121-W3H at the alternating opposed end of channel assembly 121. Note that along the air flow path, the location of the openings in the walls alternate back and forth from one end of channel assembly 121 to the other as the air passes through channel assembly 121 from channel to channel. Air flow continues in this manner in a serpentine path between alternating opposed ends of channel assembly 121 until the treated air exits channel assembly 121 at air output 121-OUT.

Referring back to FIG. 1K, it is noted that the opposed ends of each wall extend beyond the closed side 121-WS-A of wall retainer 121-WS-R. This means that when removable ultraviolet (UV) light source modules 140-1 and 150-1 are mounted to wall structure 121-WS, the wall holes 121-W1H, 121-W2H, 121-W3H, 121-W4H and 121-W5H are situated within modules 140-1 and 150-1. In other words, the wall holes 121-W1H to 121-W5H make the back and forth air flow possible from channel to adjacent channel are situated within modules 140-1 and 150-1 when PTC 120 is fully assembled as shown in FIG. 1L and FIG. 1M.

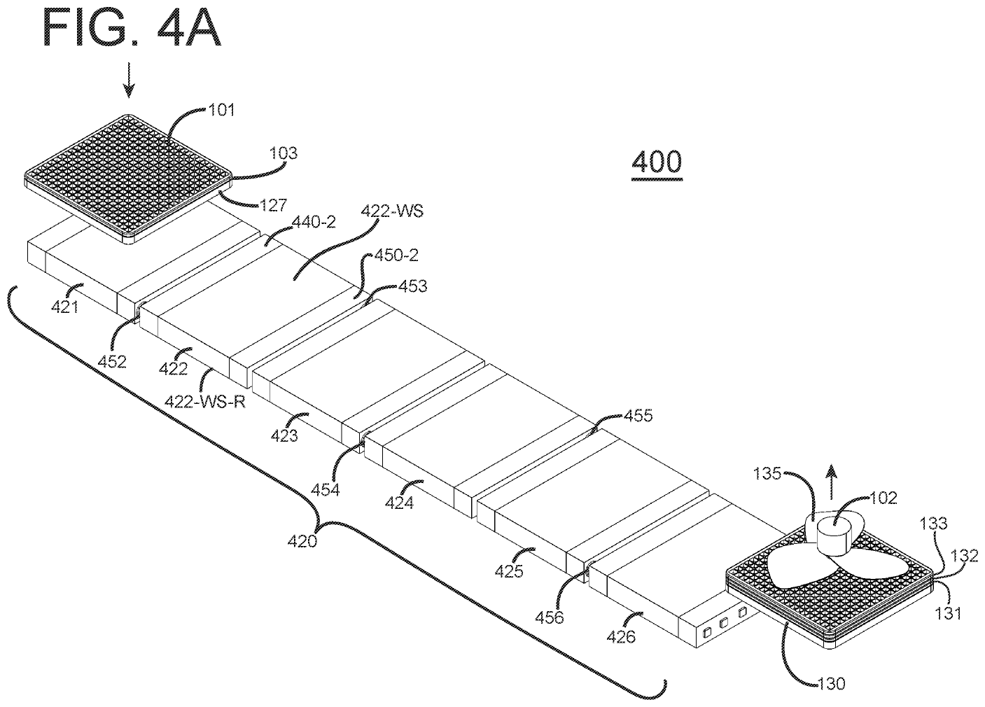

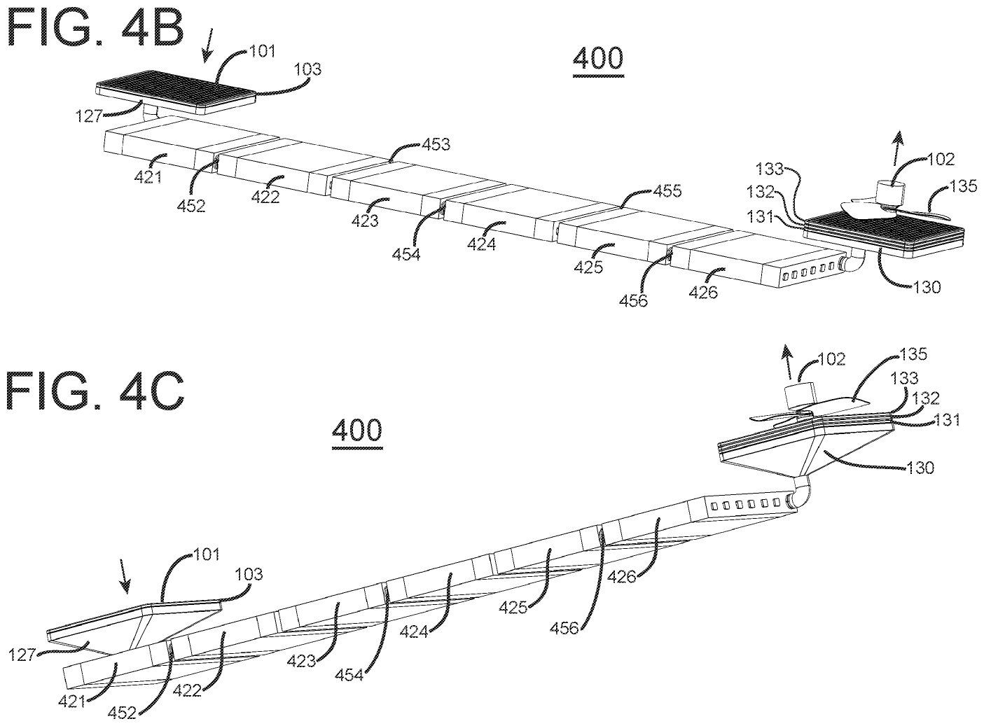

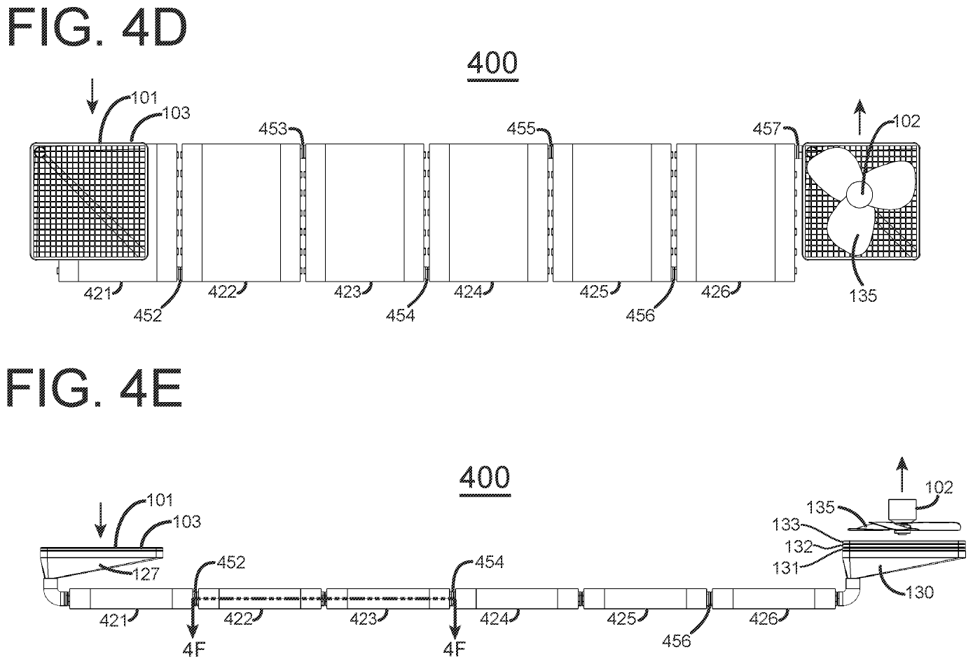

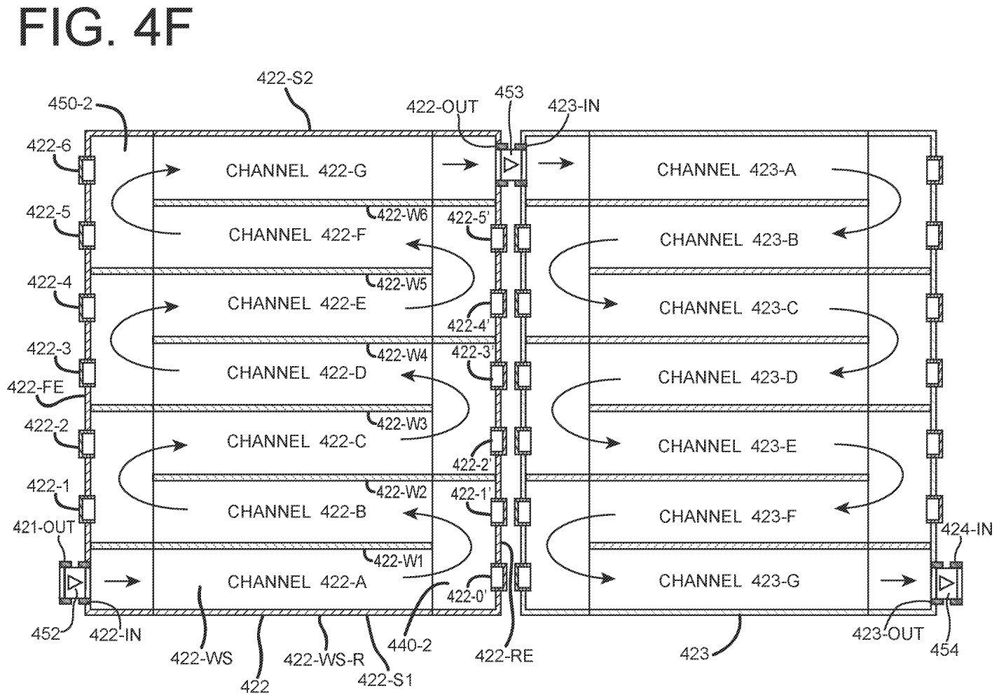

FIG. 1N is a side perspective view of channel assembly 122 that is situated below channel assembly 121 in FIG. 1L. The builder constructs channel assembly 122 in a manner similar to that of channel assembly 121 described above. It is noted that the builder need not necessarily be human, but could be robotic as well. In either case, the builder positions removable UV light source module 140-2 over the ends of the walls (not shown) of the wall retainer member 122-WS-R such that the rear 140-2R of UV light source module 140-2 is flush with wall retainer member 122-WS-R, as shown in FIG. 1N. Likewise, the builder positions removable UV light source module 150-2 over the remaining ends of the walls of the wall retainer member 122-WS-R such that the rear 150-2R of UV light source module 150-2 is flush with wall retainer member 122-WS-R, as illustrated. When channel assembly 122 is installed below channel assembly 121, such as seen in FIG. 1L, the air input 122-IN of channel assembly 122 is situated below the air output (not shown) of channel assembly 121. A pipe, valve, or one-way valve (not shown) may be used to achieve this coupling of the treated air output 121-OUT (seen in FIG. 11) of channel assembly 121 to the air input 122-IN of channel assembly 122. More details of a channel assembly connector suitable for connecting one channel assembly to another are provided in the discussion of FIGS. 4A-4F.

FIG. 1O is a cross-sectional view of channel assembly 122 of FIG. 1N taken along section line 1O-1O. FIG. 1O shows the interior of channel assembly 122 as including walls 122-W1, 122-W2, 122-W3, 122-W4 and 122-W5 that define channels 122A, 122B, 122C, 122D, 122E and 122F. The air flow path through representative channel assembly 122 is generally opposite that of channel assembly 121 of FIG. M and is now described. Straight and curved arrows illustrate the air flow path from air input opening 122-IN to air output opening 122-OUT in channel assembly 122. Since air input opening 122-IN is above the section line 1O-1O, air input opening 122-IN is not actually visible in FIG. 1M but is nonetheless marked in FIG. 1O to instruct the reader where air flows into channel assembly 122 in the general sense. If more channel assemblies are added to the PTC 120 below channel assembly 122, the air flow of each added channel assembly is generally opposite that of the channel assembly immediately above.

Air to be treated by channel assembly 122 passes through air input opening 122-IN where adjacent UV light source 122-1′ treats the air with ultraviolet (UV) light. The treated air travels along channel 122A from the end of channel 122A adjacent UV light source 122-1′ to the opposite end of channel 122A where UV light source 122-1 further treats the treated air. The treated air passes through wall hole 122-W1H and into the end of channel 122B adjacent UV light source 122-2 which further treats the air with UV light. Now the treated air travels along channel 122B from the end of channel 122B adjacent UV light source 122-2 to the opposite end of channel 122B where UV light source 122-2′ further treats the treated air. The treated air passes through wall hole 122-W2H and into the end of channel 122C adjacent UV light source 122-3′ which further treats the air with UV light.

The treated air travels along channel 122C from the end of channel 122C adjacent UV light source 122-3′ to the opposite end of channel 122C where UV light source 122-3 further treats the treated air. The treated air passes through wall hole 122-W3H and into the end of channel 122D adjacent UV light source 122-4 which further treats the air with UV light. Next the treated air travels along channel 122D from the end of channel 122D adjacent UV light source 122-4 to the opposite end of channel 122D where UV light source 122-4′ further treats the treated air. The treated air passes through wall hole 122-W4H and into the end of channel 122E adjacent UV light source 122-5′ which further treats the air with UV light.

The treated air travels along channel 122E from the end of channel 122E adjacent UV light source 122-5′ to the opposite end of channel 122E where UV light source 122-5 further treats the treated air. The treated air passes through wall hole 122-W5H and into the end of channel 122F adjacent UV light source 122-6 which further treats the air with UV light. Now the treated air travels along channel 122F from the end of channel 122F adjacent UV light source 122-6 to the opposite end of channel 122F where UV light source 122-6′ further treats the treated air. The treated air exits channel 122F and channel assembly 122 at air output opening 122-OUT. Comparing channel assembly 121 of FIG. 1M with channel assembly 122 of FIG. 1O, it is noted that while air flow internal to a channel assembly exhibits a “back and forth” pattern between channel ends as shown, the general overall air flow of channel assembly 121 is in direction D1 from air input 121-IN to air output 121-OUT as seen in FIG. 1M. Turning now to FIG. 1O, it is seen that the general overall air flow of channel assembly 122 is in direction D2 from air input 122-IN to air output 122-OUT. These flow directions D1 and D2 are opposite one another. Comparing the internal air flow paths through the channels of channel assembly 121 and channel assembly 122, those internal air flow paths are opposite one another as well in this particular embodiment. In other words, the air flow of each channel is in a direction opposite that of an adjacent channel.

In one embodiment, all of the UV light sources of pathogen treatment chamber (PTC) 120 exhibit the same UV wavelength. In a preferred embodiment wherein each channel includes respective UV light sources at the channel's opposed ends, the UV light source at one end of a channel exhibits a first wavelength while the UV light source at the opposite end of that channel exhibits a different wavelength. The wavelength of these UV light sources may be selected to act on particular pathogens. Using channel assembly 121 of FIG. 1M as an example, UV light sources 121-1, 121-2, 121-3, 121-4, 121-5 and 121-6 are selected to generate a wavelength in the UV-C Band between approximately 220 nm and approximately 250 nm, while UV light sources 121-1′, 121-2′, 121-3′, 121-4′, 121-5′ and 121-6′ are selected to generate a wavelength between approximately 250 and approximately 280 nm. In this embodiment, such UVC wavelengths are effective in reducing the SARS-CoV-2 pathogen in the air supplied to pathogen treatment chamber (PTC) 120.

Although in one embodiment channel assemblies can be stacked directly on top of one another, the currently preferred embodiment shown in FIG. 1R employs a 2-piece mating interposer assembly 170 that can connect one channel assembly to another channel assembly. Interposer 170 includes a top interposer component 161 as seen in FIG. 1P. Interposer assembly 170 further includes a bottom interposer component 162 as seen in FIG. 1Q. While the particular interposer 170 shown in FIG. 1R exhibits a square geometry, interposer 170 could also exhibit a rectangular geometry or other geometry that generally matches the geometry of pathogen treatment chamber (PTC) 120. As seen in FIG. 1P, bottom interposer component 161 exhibits a square geometry with 4 sides that include male interconnects 161A, 161B, 161C and 161D, respectively. Correspondingly, as seen in FIG. 1Q, top interposer component 162 exhibits a square geometry with 4 sides that include female interconnects 162A, 1621B, 162C and 162D, respectively. To connect bottom interposer component 161 to top interposer component 162, the male interconnects 161A, 161B, 161C and 161D are mated with the female interconnects 162A, 162B, 162C and 162D, respectively, as shown in FIG. 1R. The female interconnects are sufficiently deep to prevent dislodgement of adjacent channel assemblies due to any unintended agitation or shocks to system 100. In an alternative embodiment, bottom interposer component 161 may include the female interconnects while top interposer component 162 includes the male interconnects. The male interconnects 161A, 161B, 161C and 161D and the female interconnects 162A, 162B, 162C and 162D may also be referred to as interconnect joints. For example, when male interconnect 161A mates with female interconnect 162A, a joint is formed where one interconnect meets the other.

FIG. 15 shows a perspective view of channel assembly 121 with a top interposer component 160 and a bottom interposer component 161 thereon. FIG. 1T shows a perspective view of channel assembly 122 with a top interposer component 162 and a bottom interposer component 163 thereon. FIG. 1U shows channel assemblies 121 and 122 with bottom interposer component 161 and top interposer component 162 mated together to form interposer 170. Channel assemblies 121 and 122 connect together in this manner.

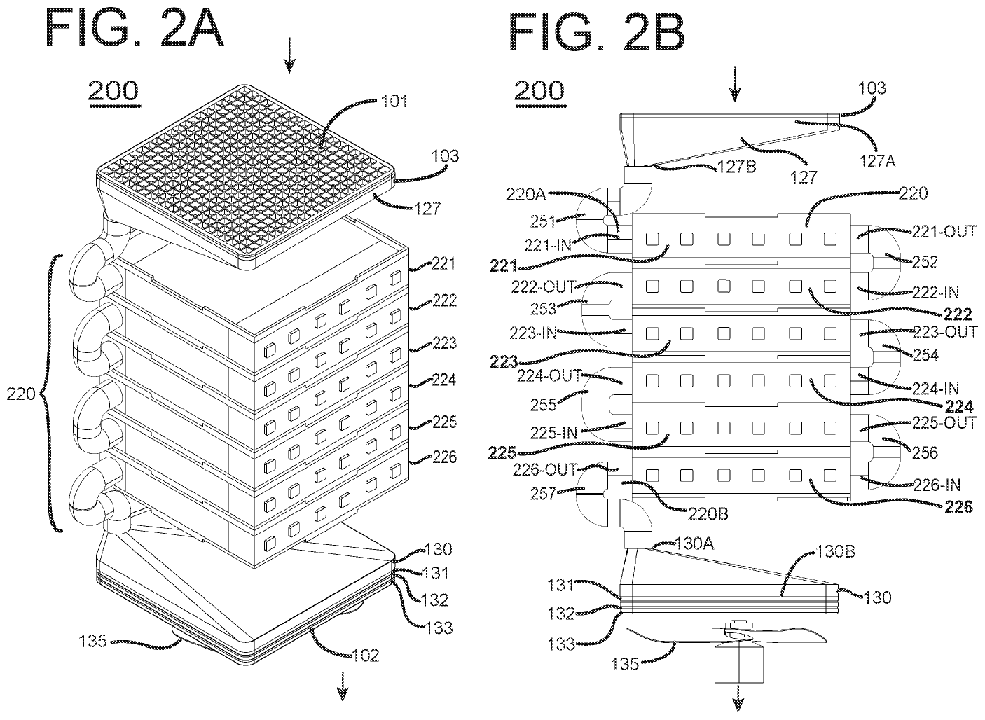

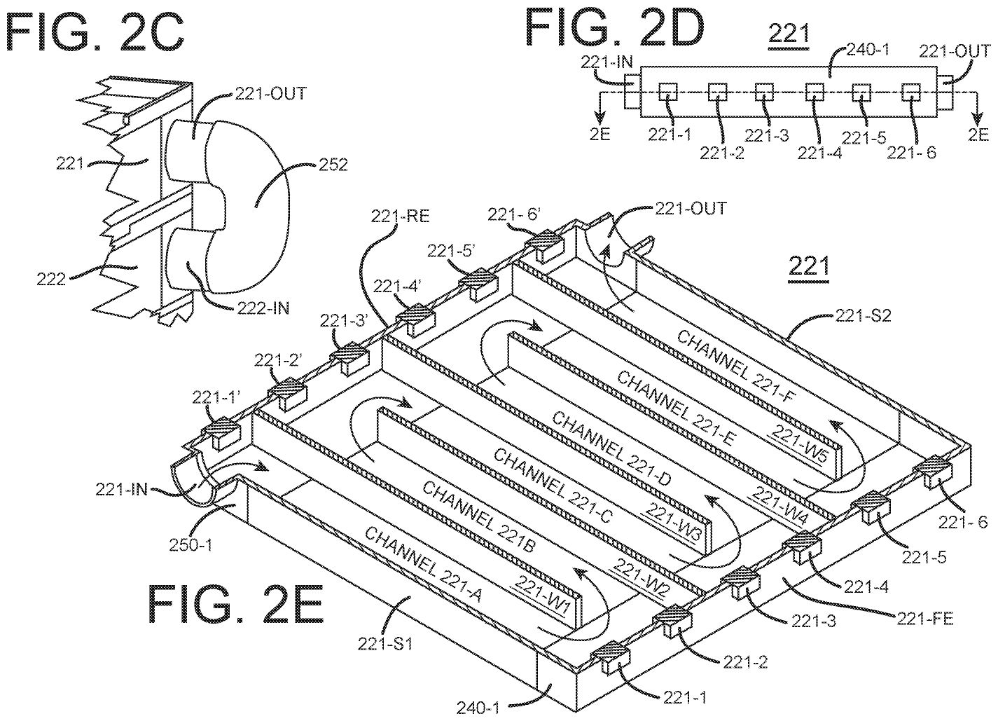

FIG. 2A shows a side perspective view of another embodiment of the disclosed air treatment system as system 200. System 200 includes many components in common with system 100 of FIGS. 1A-1U. Like numbers indicate like components when comparing these two systems, for example the components main air input 101, main air output 102, funnels 127 and 130, filters 131-133, and impeller 135. Like system 100, system 200 includes vertically stacked channel assemblies. While in one embodiment the channel assemblies of system 100 are coupled together by one-way valves, in another embodiment the channel assemblies of system 200 are coupled together by pipes. System 200 includes several components that while not identical to components of system 100 are similar to components of system 100. For example, channel assemblies 221, 222, 223, 224, 225 and 226 of system 200 of FIG. 2B are similar to channel assemblies 121, 122, 123, 124, 125 and 126 of system 100 of FIG. 1B. However, the channel assemblies 221, 222, 223, 224, 225 and 226 of system 200 are coupled together via pipes or other flexible tubes as discussed in more detail below.

FIG. 2B depicts vertically stacked channel assemblies 221-226 that couple together via connecting pipes 251-256. In one embodiment, these connecting pipes may be flexible hoses made of polyvinyl chloride (PVC) or other flexible tubular material. By way of example, pipe 251 sealably couples funnel output 127B to the input 221-IN of channel assembly 221. Pipe 251 may also be called a channel assembly connector because it connects channel assembly 221 to other structures. Since channel assembly 221 is the topmost channel assembly of the 6 channel assembly stack depicted in FIG. 2B, input 221-IN of channel assembly 221 is also the input 220-A of pathogen treatment chamber (PTC) 220.

Pipe 252 couples output 221-OUT of channel assembly 221 to input 222-IN of channel assembly 222 which is situated immediately below channel assembly 221. Referring now to FIG. 2C, a closeup view of output 221-OUT, input 222-IN, pipe 252 and the adjacent portions of channel assemblies 221 and 222 is shown. In one embodiment, output 221-OUT and input 222-IN may be pipes of the same outer diameter, while pipe 252 may exhibit an inner diameter slightly larger than the outer diameters of output 221-OUT and input 222-IN. In this manner, the opposite ends pipe 252 can snugly fit over output 221-OUT and input 222-IN to provide a light-tight and air tight seal. Moreover, output 221-OUT of channel assembly 221 and input 222-IN of channel assembly 222 are sealably coupled together. The pipe used as output 221-OUT and the pipe used as input 222-IN may also be referred to as fittings and can be made of rigid material as well as flexible material. The remaining channel assemblies 223, 224, 225, 226, 227 and funnel 130 are connected together in a similar manner to that described above for channel assemblies 221 and 222. Pipes 253, 254, 255, 256 and 257 may be used to form these channel assembly to channel assembly connections or connections to other structures such as funnel 130. These pipes 251, 252, 253, 254, 255, 256 and 257 may include one-way valves to assure that air flows in the direction shown by the arrows of FIG. 2B to avoid air backflow. Since pipes 251, 252, 253, 254, 255 256 and 257 connect the channel assemblies to one another or other structures, these pipes may also be referred to as channel assembly connectors.

FIG. 2D depicts a closeup plan view of removable UV light source module 221 with section line 2E-2E. FIG. 2E is a cross sectional perspective view of channel assembly 221 taken along section line 2E-2E of FIG. 2D Both input 221-IN and output 221-OUT are seen in FIG. 2E. Channel assembly 221 includes channels 221-A, 221-B, 221-C, 221-D, 221-E and 221-F that are formed between walls 221-W1, 221-W2, 221-W3, 221-W4 and 221-W5 in a manner similar to that depicted for channel assembly 121 in FIG. 1M. However, in the particular embodiment depicted in FIG. 2E each wall terminates before reaching, and does not extend into, the UV light source module adjacent one end of each wall. This provides wall open regions indicated by U-shaped arrows in each channel where air flow changes direction as depicted. In comparison to the wall holes 121-W1H, 121-W2H, . . . 121-W5H of FIG. 1M that form partially open wall ends, the open regions indicated by U-shaped arrows in FIG. 2E are completely open in one embodiment. For clarity, front end 221-FE is the end of channel assembly 221 where UV light sources 221-1, 221-2, . . . 221-6 are located, whereas the opposite end of channel assembly 221 is considered to be the rear end 221-RE of channel assembly 221 where the remaining UV light sources 221-1′, 221-2′, . . . 221-6′ are located. In contrast, opposed sides 221-S1 and 221-S2 are the sides of channel assembly 221 running along the two outer opposed channels 221A and 221F, respectively.

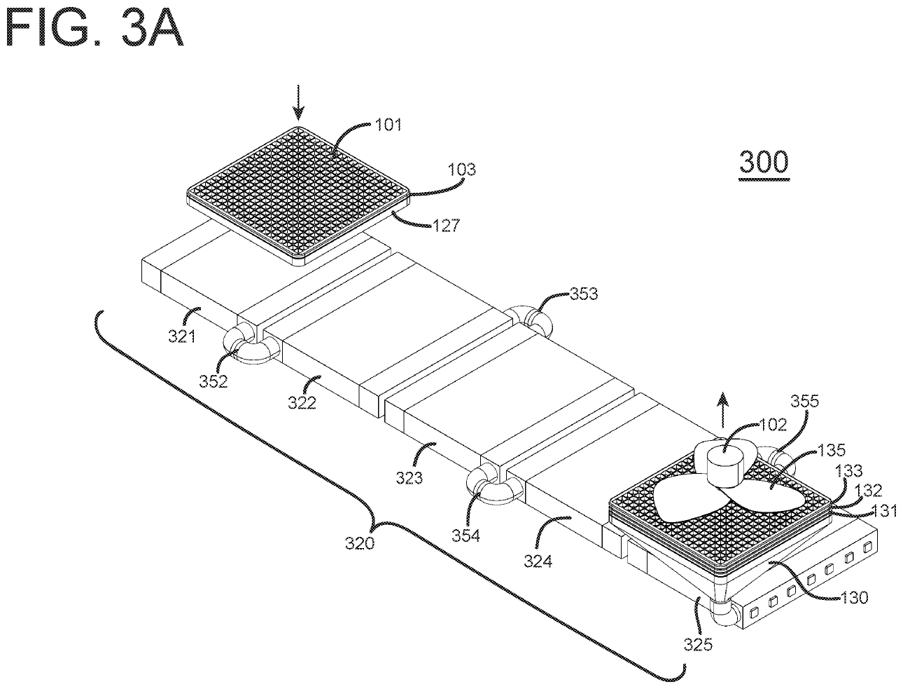

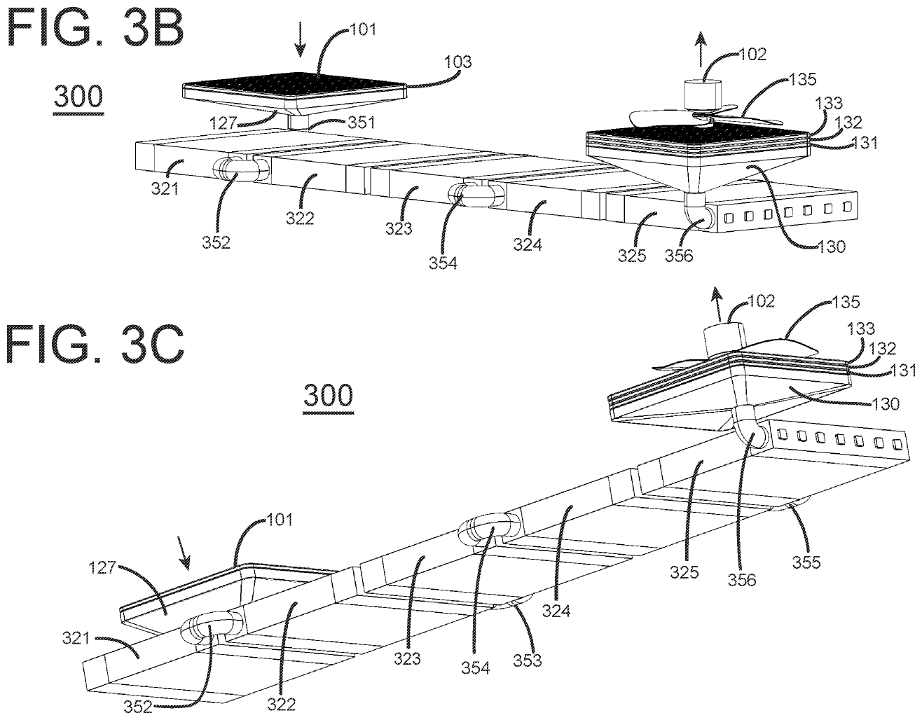

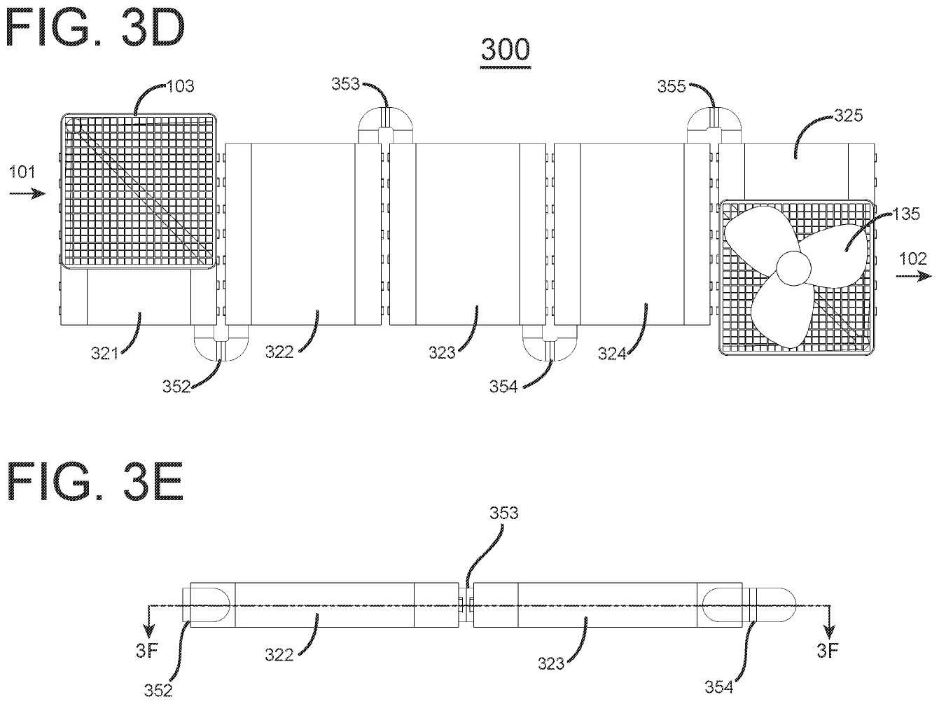

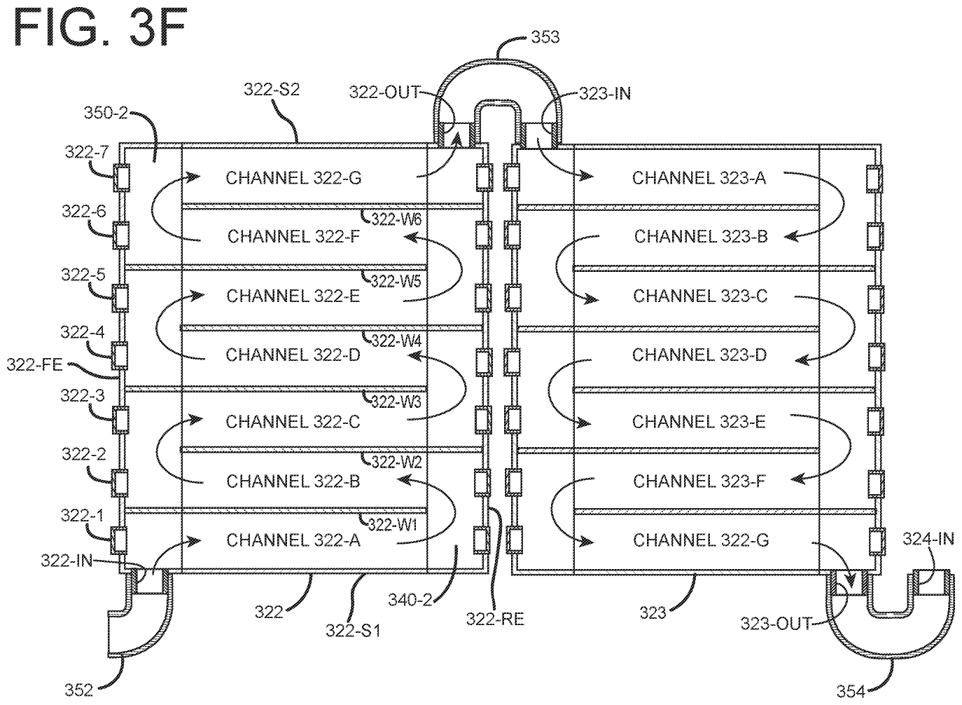

FIG. 3A depicts an air treatment system 300 that employs substantially the same channel assemblies and pipe connectors as vertically stacked air treatment system 200, except that system 300 configures the channel assemblies end-to-end in the same plane in a horizontally stacked relationship. More specifically, system 300 of FIG. 3A includes many components in common with system 200 of FIGS. 2A-2E and system 100 of FIGS. 1A-1U. Like numbers indicate like components when comparing these systems, for example the components main air input 101, main air output 102, funnels 127 and 130, filters 131-133, and impeller 135. FIG. 3A shows an embodiment of system 300 wherein the channel assemblies are configured in a horizontally stacked, spaced-apart relationship.

Like system 200, system 300 includes connective pipes such as flexible pipes, rigid pipes, flexible hoses, or combinations of these elements to connect one channel assembly to an adjacent channel assembly, as seen in FIG. 3A, FIG. 3B and FIG. 3C. System 300 includes several components that while not identical to components of system 200 are similar to components of system 200. For example, channel assemblies 321, 322, 323, 324 and 325 of system 300 of FIG. 3A-3D are similar to channel assemblies 221, 222, 223, 224 and 225, respectively, of system 200 of FIG. 2B. These channel assemblies are coupled together via pipes or other flexible tubes as discussed in more detail below. However, whereas the channel assemblies 221-226 of FIG. 2B have their inputs and outputs on a common rear end of each channel assembly (e.g. input 221-IN and output 221-OUT of channel assembly 221 in FIG. 2E), the inputs and outputs of channel assemblies 321-325 of FIG. 3D are on opposite sides of each channel assembly. Moreover, in the particular embodiment of channel assembly 221 depicted in FIG. 2E, the input 221-IN and the output 221-OUT are situated in the same removable ultraviolet (UV) light source module 250-1. In contrast, in the particular channel assembly 322 depicted in FIG. 3F (which is a cross section of channel assemblies 322 and 323 of FIG. 3E taken along section line 3F-3F), input 322-IN and output 322-OUT of channel assembly 322 are situated on opposite sides and opposite ends of channel assembly 322, as discussed in more detail below. Input 322-IN is situated at one side of removable UV light source module 350-2, while output 322-OUT is situated at an opposite side of removable UV light source module 340-2. For clarity, the end of channel assembly 322 where UV light sources 322-1, 322-2, . . . 321-7 are located is considered to be the front end 321-FE of channel assembly 322, whereas the opposite end of channel assembly 322 is considered to be the rear end 321-RE of channel assembly 322 where the remaining UV light sources (not labelled) are located. Moreover, the side of channel assembly 322 running along channel 322-A and the side of channel assembly 322 running along channel 322-G may be referenced as the opposed sides 322-S1 and 322-S2, respectively, of channel assembly 322.

Returning to FIG. 3A, this view of system 300 shows that connecting pipe 352 connects the output of channel assembly 321 to the input of channel assembly 322. In more detail, FIG. 3F shows a portion of connecting pipe 352 that couples to input 322-IN. Connecting pipe 352 is dimensioned to exhibit an inner diameter slightly larger than the outer diameter of input 322-IN so that connecting pipe 352 snugly fits over input 322-IN to form a light tight and air tight seal. In this manner, air provided to connecting pipe 352 by channel assembly 321 flows into input 322-IN and channel 322-A as indicated by arrows in FIG. 3F. In FIG. 3F, walls 322-W1 through 322-W6 form channels 322-A through 322-G, as shown. A UV light source is situated at each end of each channel 3222-A through 322-G. For example, UV light sources 322-1 through 322-7 are situated at one end (i.e. the front end) of channels 322-A through 322-G, while other UV light sources (not specifically numbered) are situated at the opposed end, i.e. the rear end, of each of these channels in channel assembly 322.

FIG. 3F also shows a complete connecting pipe 353, the ends of which couple to channel assembly 322 output 322-OUT and channel assembly 323 input 323-IN in a manner similar to that discussed above with reference to connecting pipe 352 of FIG. 3F and connecting pipe 252 of FIG. 2C. FIG. 3F further shows a complete connecting pipe 354, one end of which couples to channel assembly output 323-OUT.

FIG. 4A is a top perspective view of another embodiment of the disclosed air treatment system 400 wherein the channel assemblies are arranged end-to-end as modules in the same plane in a horizontal configuration. System 400 employs substantially the same channel assemblies and channel assembly connectors as vertically stacked air treatment system 100, except that system 400 configures the channel assemblies horizontally end-to-end in a spaced-apart relationship. More specifically, system 400 includes many components in common with system 100 of FIGS. 1A-1U. Like numbers indicate like components when comparing these systems, for example the components main air input 101, main air output 102, funnels 127 and 130, filters 131-133, and impeller 135.

System 400 includes channel assemblies 421, 422, 423, 424, 425 and 426 that are coupled together by channel assembly connectors 452, 453, 454, 455 and 456 (partially visible in FIG. 4A). Channel assemblies 421, 422, 423, 424, 425 and 426 together form a modular pathogen treatment chamber (PTC) 420. More or fewer channel assemblies may be connected to one another as modules as taught herein to provide a desired amount of pathogen treatment within pathogen treatment chamber 420. Representative channel assembly 422 includes wall structure 422 as well as UV light source modules 440-2 and 450-2. FIG. 4B is another top perspective view of system 400, while FIG. 4C provides a bottom perspective view of system 400.

FIG. 4D is a top plan view of system 400 that shows channel assemblies 421-426 that are coupled together by channel assembly connectors 452-457 situated between the channel assemblies. Channel assembly connectors 452-457 provide light tight and air tight connection between channel assemblies. FIG. 4E is a side plan view of system 400 that includes a section line 4F-4F. FIG. 4F shows the cross section indicated by section line 4F-4F of FIG. 4E. The cross section shown in FIG. 4F reveals the interior of channel assemblies 422 and 423 as well as representative channel assembly connectors 452, 453 and 454. In the embodiment of FIG. 4F, channel assembly connectors are implemented as one-way valves that allow air flow in the direction indicated by the arrow in each connector 452, 453 and 454 and the remainder of FIG. 4F in general. Alternatively, channel assembly connectors 452, 453 and 454 may be open pipes that pass air without directional restriction as long as the channel assembly connectors selected are light tight and air tight.

As seen in FIG. 4F, channel assembly 422 includes a wall retainer 422-WS-R with integral walls 422-W1, 422-W2, 422-W3, 422-W4, 422-W5 and 422-W6 that define channels 422-A, 422-B, 422-C, 422-D, 422-E, 422-F and 422-G. In this manner, wall retainer 422-WS-R is similar to wall retainer 121-WS-R illustrated in FIGS. 1K and 1L. Channel assembly 422 further includes removable ultraviolet (UV) light source module 440-2 and removable ultraviolet (UV) light source module 440-2 that fit over the ends of walls 422-W1, 422-W2, . . . 422.W6, as shown. UV light source module 450-2 includes UV light sources 422-1, 422-2, . . . 422-6 at one end of channels 442A, 442B, . . . 442G, respectively. Similarly, UV light source module 440-2 provides respective UV light sources 422-1′, 422-2′, . . . 422-5′ at the remaining ends of channels 442-B, 422-C, . . . 442F, as seen in FIG. 4F. For clarity, the end of channel assembly 422 where UV light sources 422-1, 422-2, . . . 421-6 are located is considered to be the front end 422-FE of channel assembly 4322, whereas the opposite end of channel assembly 422 is considered to be the rear end 422-RE of channel assembly 422 where the remaining UV light sources 422-0′, 422-1′, . . . 421-5′ are located. Moreover, the side of channel assembly 422 running along channel 422-A and the side of channel assembly 422 running along channel 422-G may be referenced as opposed sides 422-S1 and 422-S2, respectively, of channel assembly 422.

Air from the output of channel assembly 421 (not shown in FIG. 4F) enters channel 422-A of channel assembly 422 via channel assembly connector 452 that attaches to input 422-IN. Air follows a serpentine path through channels 422-A, 422-B, . . . 422-G. Each time the air passes an interior end of a channel wall, the air is treated by 2 different UV light sources. For example, as the air rounds the interior end of wall 422-W1 at UV light source module 440-2, the air is treated by adjacent UV light sources 422-0′ and 422-1′. After exposure to UV light, the air thus treated may be referred to as “treated air”. The longer the serpentine path, the more time the pathogen in the air is exposed to UV light, and thus the more pathogen is inactivated, neutralized or otherwise ameliorated. Continuing with another example, as the now “treated air” rounds the interior end of wall 422-W2 at UV light source module 450-2, the air is further treated by adjacent UV light sources 422-1 and 422-2. Moreover, as the now further treated air rounds the interior end wall 422-W3 at UV light source module 440-2, the treated air is still further treated by adjacent UV light sources 422-2′ and 422-3′. Further treatment of the treated air continues in this manner within channel assembly 422 along the assembly's serpentine path until the treated air exits channel 422-G via output 422-OUT.

In the particular embodiment that FIG. 4F depicts, the output 422-OUT of channel assembly 422 couples to the input 423-IN of channel assembly 423 via channel assembly connector 453. This effectively adds another stage, i.e. module, of air treatment to the treatment already provided by channel assembly 422 and channel assembly 421. Channel assembly connector 453 may be a one-way valve in one embodiment, or a passthrough coupler tube in another embodiment that allows air treated by channel assembly 422 to freely flow through connector 453 to channel assembly 423 for further treatment. The channel assembly connectors 452 and 454 may take the same form as channel assembly connector 453 discussed above. Channel assemblies 424, 425 and 426 exhibit internal wall configurations and coupling together by channel assembly connectors that are similar to that discussed above with reference to channel assemblies 422 and 423. Each channel assembly through which the treated air passes further reduces the viral load exhibited by the treated air. In this manner, the user may select a number of modules, i.e. channel assemblies, desired to be coupled together in series in a pathogen treatment chamber (PTC) for use in a particular environment to treat or purify the treated air to an acceptable level of reduced pathogen.

FIG. 5A is a perspective view of a removable UV light source module 140-1 that includes a UV LED strip 502 situated on front end 140-1E of the module. FIG. 5B is a plan view of removable UV light source module 140-1 showing front end 140-1E on which UV LED strip 502 is mounted by screws 504, 506 and 508 that hold UV LED strip 502 to front end 140-1E. UV LED strip 502 includes a printed circuit board or flex circuit board with UV light sources 121-1, 121-2, 121-3, 121-4, 121-5 and 121-6 situated thereon as shown. UV light sources 121-1, 121-2, 121-3, 121-4, 121-5 and 121-6 couple across positive power rail 510 and negative power rail 512. Positive power rail 510 couples to opposed end terminals 510A and 510B. Negative power rail 512 couples to opposed end terminals 512A and 512B.

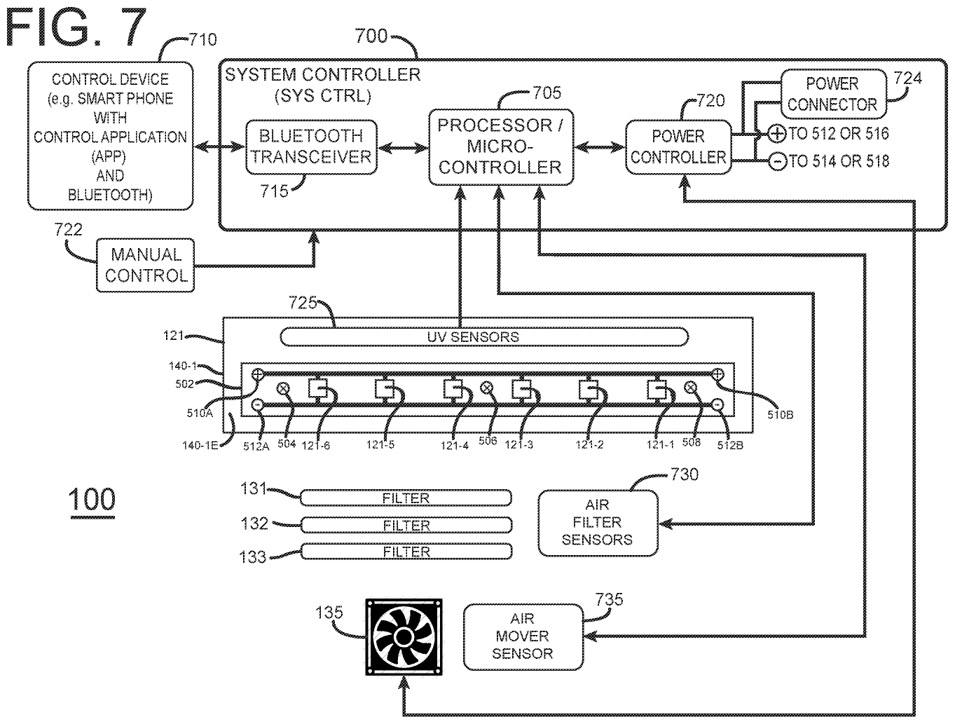

FIG. 5C is a block diagram of the UV LED strips 502 of respective UV light source modules 140-1 of three representative channel assemblies 121, 122 and 123. Like numbers are used to indicate like components in channel assemblies 121, 122 and 123. Positive end terminals 510B of channel assemblies 121, 122 and 123 couple together and to a positive voltage source (shown in FIG. 7). Negative end terminals 510B of channel assemblies 121, 122 and 123 couple together and to a negative voltage source (shown in FIG. 7). In this manner, UV light sources 121-1, 121-2, 121-3, 121-4, 121-5 and 121-6 are appropriately biased to emit UV light as the pathogen passes by these light sources. When one or more UV light sources fail in a UV LED strip 502, the entire UV light source module 140-1 containing the failed UV light sources may be replaced with a new UV light source module 140-1. To make such an exchange easier, the positive terminals 510A, 510B and the negative terminals 512A, 512B of each removable UV light source module 140-1 may be replaced with a plug-in power connector that couples to plug-in power connector 724 shown in FIG. 7. In this manner power controller 720 supplies power to removable UV source modules 140-1.

FIG. 6A is a perspective view of air treatment system 100 shown with four frame members 601, 602, 603 and 604 situated at the four corners of system 100, respectively. Frame members 601, 602, 603 and 604 may be referred to as plastic or aluminum right angle extrusions, extruded angles, angle equal legs, angle irons or L-shaped members. Frame members 601, 602, 603, 604 employ screws (not shown) that screw each of these frame members to adjacent grill 103, adjacent funnel 127, adjacent channel assemblies 121-126, adjacent funnel 130 and optionally adjacent filters 131, 132 and 133.

FIG. 6B is a perspective view of air treatment system 100 with a housing enclosure 610 situated surrounding the 4 sides thereof. In one embodiment, housing 610 includes sides 610A, 610B, 610C and 610D that together enclose four sides of system 100 except for open top 612 and open bottom 614 which are left open to enable air flow through system 100. In this embodiment, housing 610 is a rectangular parallelepiped with open ends 612 and 614. These open ends of housing 610 allow passage of air from main air input 101 to main air output 102. Housing 610 may be screwed to frame members 601-604 to hold housing 610 in position on frame members 601-604 using aligned screw holes (not shown) in housing 610 and frame members 601, 602, 603 and 604 below.

FIG. 6C is a perspective view of system 100 showing system controller (SYS CTRL) 700 located on the side of funnel 130 in a position free of other components. It is also possible to locate system controller 700 at other locations in the system such as on funnel 127, for example. FIG. 6D shows an embodiment of system 100 wherein housing 610 is divided into 3 sub-housings 610-1, 610-2 and 610-3. Sub-housing 610-1 is an upper housing section wraps around the 4 sides and of the uppermost portion of system 100 to enclose the 4 sides thereof as shown. Sub-housing 610-2 is a mid-housing section that wraps around the 4 ides of the middle portion, i.e. mid-section, of system 100 to enclose the 4 sides thereof as shown. Sub-housing 610-3 is a lower housing section that wraps around the 4 ides of the lowermost portion of system 100 adjacent funnel 130 to enclose those 4 sides as shown. In one embodiment, sub-housing 610-3 includes a door 620 with a latch 625. Door 620 pivotably attaches to sub-housing 610-3 via a hinge 630. When the user engages latch 625, door 620 swings open about hinge 630 to provide user access to removable filters 131, 132 and 133. In this manner, the user may easily replace any of filters 131, 132 and 133 when a filter reaches end of life. System controller 700 is also visible on funnel 130 when door 620 is open.

FIG. 7 is a high level block diagram of one embodiment of air treatment system 100. System 100 includes a system controller 700 that guides the overall operation of system 100. System controller 700 includes a processor or microcontroller 705. Processor 700 can receive commands from a control device 710 such as a smartphone that communicates commands to processor 705 via a radio frequency transceiver 715, such as a Bluetooth transceiver, as shown. Other radio frequency protocols such as WiFi may be used to communicate between control device 710 and processor 705. Control device 710 may include a control application (APP) that aids in monitoring and controlling the operation of system 100. System controller 700 includes a power controller 720 that couples to processor 705 to receive commands from, and send information to, processor 705. Power controller 720 distributes power to other devices in system 100 such as channel assembly 121 and air mover 135. A manual control 722 couples to system controller 700 to manually control air treatment system 100 without using control device 710. For example, manual control 722 may include an on/off power switch for turning the system on and off. Manual control 722 may also include a system reset switch/button (not shown) that instructs processor 705 to reset, i.e. reboot.

Air treatment system 100 includes representative channel assembly 121 including UV LED light sources 121-1, 121-2, 121-3, 121-4, 121-5 and 121-6 to which power controller 720 provides power via positive terminals 510A or 510B and negative terminals 512A or 512B. Channel assembly 121 includes ultraviolet (UV) sensors 725 that sense the UV light emitted by UV light sources 121-1, 121-2, 121-3, 121-4, 121-5 and 121-6. UV sensors 725 couple to processor 705 to inform processor 705 of the current light output level of the UV light sources. In this manner, processor 705 knows the light output level of UV sensors 725 and can determine if one or more of the UV light sources is failing and requires replacement. In one embodiment, UV sensors 725 include one UV sensor per UV light source. Each UV sensor sends status information such as UV output good, UV output low, or no UV output back to processor 705. In this manner, processor 705 receives UV light source status information on a UV light source by UV light source basis. It is noted that FIG. 7 illustrates system 100 principally from an electrical high level schematic viewpoint and does not show all mechanical structures that have already been discussed above. For example, FIG. 7 shows electrical components of channel assembly 121 without illustrating the mechanical wall structures, channels and serpentine air flow path through channel assembly 121 previously shown and described.

Air treatment system 100 further includes air filter sensors 730 that couple to processor 705. In one embodiment, air filter sensors 730 include a respective air filter for each of filters 131, 132 and 133. Air filter sensors 730 sense air filter performance and send air filter performance status information back to processor 705 for each of filters 131, 132 and 133. Air treatment system 100 further includes an air mover sensor 735 that couples to processor 705. Air mover sensor 735 monitors the performance of air mover 735 and sends fan airflow performance information back to processor 705. For example, air mover sensor 735 may send air flow volume per unit time information back to processor 705.

A control application (APP) in control device 710 may facilitate wireless control of the operation of air treatment system 100. In one embodiment, the control application communicates with processor 705 to turn on and off system 100. The control application may also communicate with processor 705 to turn on and off the UV light emitters of the channel assemblies 121, 122, . . . 126. The control application may further communicate with processor 705 to control the speed/velocity of air mover 135 to manage the volume of air filtered by system 100. The control application may optionally notify the user when 1) any or all of the filters 131-133 need replacement, 2) air mover 135 is not properly functioning, and 3) any or all of the UV light sources are not functioning properly. Processor 705 monitors UV sensors 725, air filter sensors 730 and air mover sensor 735 to determine the above 3 events that may optionally be transmitted by processor 705 via transceiver 715 to the user of control device 710 to keep the user apprised of current system performance status. The control application may also enable the user to register their air treatment system with the manufacturer or other entity for warranty purposes. The control application may further inform the user with respect to the volume of air filtered during the day since the time of system turn on. The control app may alternatively enable the user to inform the manufacturer or other entity with respect to requests for service appointment and ordering replacement filters.

In one embodiment, processor 705 is configured to regulate the rotation rate of air mover 135 such that the velocity of air moving in the air flow path through a channel assembly, or the velocity of air moving through a total air flow path formed by multiple serially connected channel assemblies, provides at least approximately 30 seconds of exposure of the pathogen in the air flow (i.e. pathogen exposure time) to UV light at the wavelengths described above. In another embodiment, processor 705 is configured to regulate the air velocity provided by air mover 135 such that the pathogen exposure time is approximately 15 seconds to approximately 60 seconds. Channel assemblies can be serially stacked vertically (such as in FIGS. 1A-1U and FIGS. 2A-2E) or can be serially stacked horizontally (such as in FIGS. 3A-3F and FIGS. 4A-4F) to provide a total air flow path length that is longer than a single channel assembly can provide alone. The term “serially stacked” refers to connecting the air output of a first channel assembly to the air input of a second channel assembly such that the total airflow path length is the aggregate of the path lengths of the first and second channel assemblies. The air input of a third channel assembly can be connected to the air output of the second channel assembly to further increase the total air flow path length that the pathogen treatment chamber (PTC) provides. Four, five and even more channel assemblies can be serially aggregated in this manner to increase the total air flow path length and consequently the total amount of time to which the pathogen in the air flow is exposed to UV light. Adding more channel assemblies is this serial fashion can significantly increase the total air flow path length and consequently may increase the total time that the pathogen in the total air flow path is exposed to UV light to provide a more complete neutralization of the pathogen.

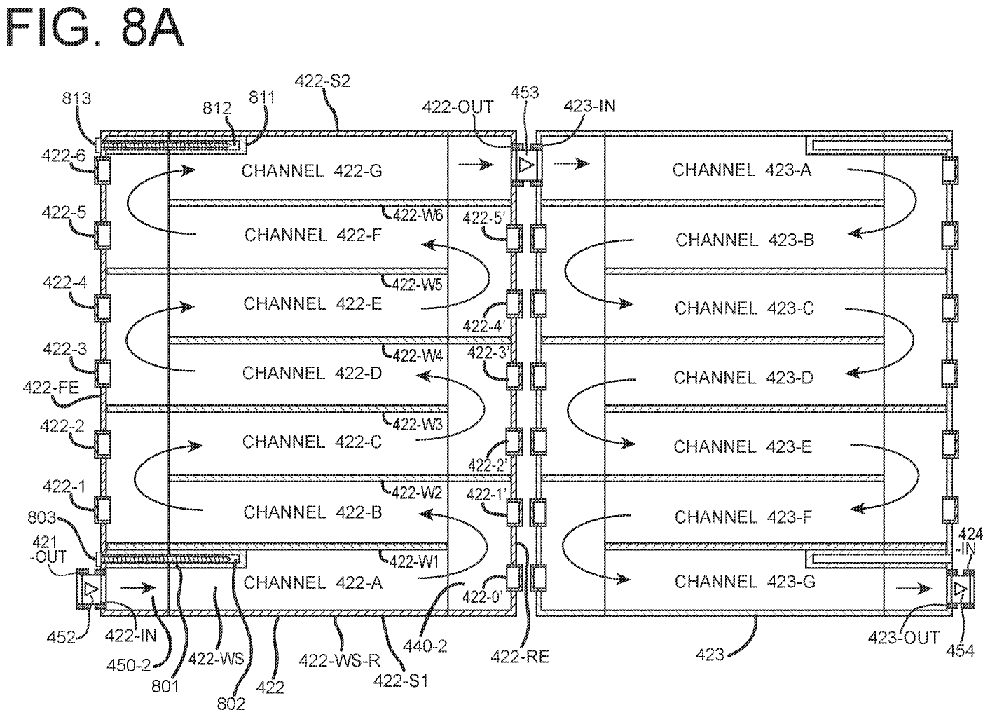

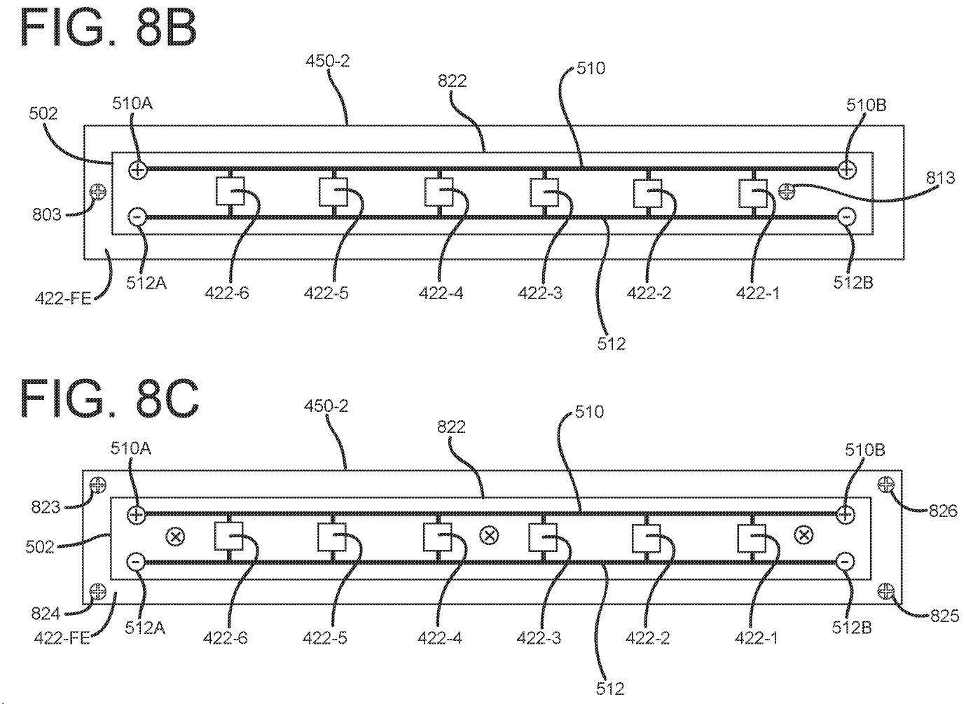

As described above, the two UV LED light source modules associated with each channel assembly are removably attached to that channel assembly. For example, each channel assembly may include multiple screw receivers with holes that receive respective screws that screw into these respective screw receivers situated on selected channel walls. FIG. 8A is similar to the cross section of channel assemblies with removable UV light source modules depicted in FIG. 4F, except that FIG. 8A shows screws and screw receivers that hold the removable UV light source modules to an associated channel assembly. Like numerals indicate like elements when comparing the channel assembly structures 422 and 423 of FIG. 8A with the channel assembly structures of FIG. 4F.

In more detail, FIG. 8A shows channel assemblies 422 and 423 wherein channel assembly 422 includes a screw receiver 801 situated on wall 422-W1 at channel 422-A. Channel assembly further includes a screw receiver 811 situated on opposed side 422-S2 at channel 422-G. Opposed side 422-S2 may be considered to be an outer wall of channel assembly 422. Screws 803 and 813 thread into screw receiver holes 802 and 812, respectively, to hold UV light source module 450-2 to wall structure 422-WS. In actual practice, channel assembly 422 may further include a screw receiver at the remaining end of channel 422-G and a screw receiver at the wall 422-W1 adjacent UV light source 422-1′ to hold UV light source module 440-2 to wall structure 422-WS. Respective screws screw into these two screw receivers to hold UV light source module 440-2 to wall structure 422-WS. More than two screws/screw receivers can be used per UV light source module to increase sealing of these structures together.

FIG. 8B shows a simplified view of front end 422-FE of FIG. 8A that includes a UV light strip 822 similar to UV light strip 502 shown in FIG. 5B. UV light strip 822 includes UV light sources 422-1, 422-2, 422-3, 422-4, 422-5 and 422-6. Screws 803 and 813 of FIG. 8B screw through respective holes in front end 422-FE of UV light source module 450-2 and into screw receivers 801 and 811 aligned therewith internal to UV light source module 450-2 of FIG. 8A.

FIG. 8C shows an alternative embodiment that employs 4 screws 823, 824, 825 and 826 that screw through respective holes adjacent the corners of front end 422-FE of UV light source module 450-2 and into 4 respective screw receivers (not shown) aligned therewith internal to UV light source module 450-2 of FIG. 8A.

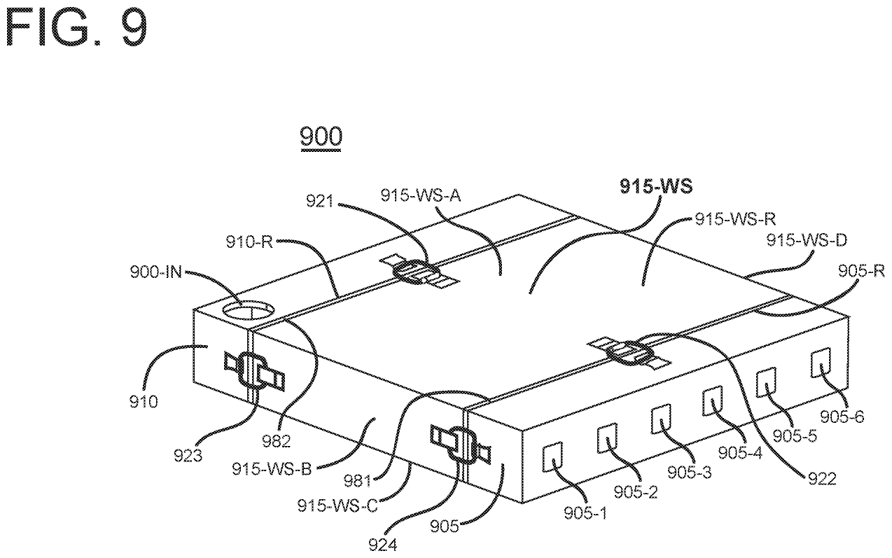

FIG. 9 shows a channel assembly 900 that employs an alternative attachment mechanism for removably attaching UV light source modules 905 and 910 to wall structure 915-WS via toggle latch fasteners. Toggle latch fasteners are also called toggle-latch clamp fasteners. Channel assembly 900 of FIG. 9 includes an air input 900-IN and is similar in structure to channel assembly 122 of FIG. 1N. More specifically, UV light source modules 905 and 910 are respectively similar to light source modules 140-2 and 150-2 of FIG. 1N. Wall structure 915-WS including its wall retainer member 915-WS-R are similar to wall structure 122-WS and its wall retainer 122-WS-RW, respectively. Wall structure retainer member 915-WS-R is a parallelepiped having 4 closed sides 915-WS-A, 915-WS-B, 915-WS-C and 915-WS-D, of which sides 915-WS-A and 915-WS-B are fully visible in FIG. 9. Wall structure retainer member 915-WS includes 2 opposed open ends that are not visible in FIG. 9, but which face rear end 905-R and 910-R, respectively, of light source modules 905 and 910. One of the open ends of wall structure retainer member 915-WS faces the rear end 905-R of UV light source module 905 while the remaining opposed open end of wall structure retainer member 915-WS faces the rear end 910-R of UV light source module 910.

One type of fastener that is suitable for removably attaching UV light source modules 905 and 910 to wall structure retainer 915-WS-R is a toggle latch. In the channel assembly 900 embodiment depicted in FIG. 9, two of such toggle latches are employed on each of the 4 sides 915-WS-A, 915-WS-B, 915-WS-C and 915-WS-D, of which only sides 915-WS-A and 915-WS-B are visible in this particular view. A toggle latch includes a base plate with lever and attached loop that engages a separate catch plate. The base plate with lever and attached loop are mounted on a first member which is to be removably fastened to a second member. The catch plate is mounted on the second member and engages the loop during fastening of the first member to the second member. In the particular latch arrangement of FIG. 9, 2 toggle latches are employed on wall structure retainer member side 915-WS-A, namely a toggle latch 921 that removably connects UV light source module 910 to side 915-WS-A, and a toggle latch 922 that removably connects UV light source module 905 to side 915-WS-A. FIG. 9 further shows 2 toggle latches that are employed on wall structure retainer member side 915-WS-B, namely a toggle latch 923 that removably connects UV light source module 910 to side 915-WS-B, and a toggle latch 924 that removably connects UV light source module 905 to side 915-WS-B. Side 915-WS-C and side 915-WS-D (not completely visible) include fastener arrangements similar to the fastener arrangements on side 915-WS-A and 915-WS-B, respectively, and operate in the same manner. Channel assembly 900 may employ silicone gaskets 981 and 982 similar to gaskets 181 and 182 of FIG. 1E to removably seal UV light source modules 905 and 910 to wall structure retainer member 915-WS-R to achieve air tightness in the air path through channel assembly 900. It is noted that the higher the latching force that the toggle latches 921-924 provide, the more that gaskets 181 and 182 compress to better seal the serpentine air flow path of channel assembly 900.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. An air treatment apparatus, comprising:
a housing including a main air input and a main air output;
at least one air mover situated within the housing to move air in an air flow path from the main air input to the main air output;
at least one filter situated in the air flow path; and
a pathogen treatment chamber (PTC) situated within the housing and intersecting the air flow path, the PTC including:
a first channel assembly of parallel channels that are situated side-by-side one another, each channel including first and second opposed ends, the first channel assembly including:
a first removable ultraviolet (UV) light source module situated spanning the first opposed ends of the first channel assembly of parallel channels and including a respective ultraviolet emitter exhibiting a first wavelength adjacent each first opposed end of the first channel assembly;
a second removable ultraviolet (UV) light source module situated spanning the second opposed ends of the first channel assembly of parallel channels and including a respective ultraviolet emitter exhibiting a second wavelength adjacent each second opposed end of the second channel assembly;
an air input opening situated at one outermost channel of the first channel assembly;
an air output opening situated at an opposed outermost channel of the first channel assembly;
the first channel assembly of parallel channels including wall members with openings at respective alternating opposed ends of the first channel assembly to form a serpentine path through the first channel assembly from the air input opening to the air output opening thereof.

2. The air treatment apparatus of claim 1, wherein the pathogen treatment chamber (PTC) further includes a second channel assembly of parallel channels that are situated side-by-side one another, each channel of the second channel assembly including first and second opposed ends, the second channel assembly of parallel channels being situated adjacent and parallel to the first channel assembly of parallel channels.

3. The air treatment apparatus of claim 2, wherein the second channel assembly of parallel channels includes:
a third removable ultraviolet (UV) light source module situated spanning the first opposed ends of the second channel assembly of parallel channels and including a respective ultraviolet emitter exhibiting the first wavelength adjacent each first opposed end of the second channel assembly;
a fourth removable ultraviolet (UV) light source module situated spanning the second opposed ends of the second channel assembly of parallel channels and including a respective ultraviolet emitter exhibiting the second wavelength adjacent each second opposed end of the second channel assembly;
an air input opening situated at one outermost channel of the second channel assembly of parallel channels;
an air output opening situated at an opposed outermost channel of the second channel assembly of parallel channels;
the second channel assembly of parallel channels including wall members with openings at respective alternating opposed ends of the second channel assembly to form a serpentine path through the second channel assembly from the air input opening to the air output opening thereof.

4. The air treatment apparatus of claim 3, wherein the air output opening of the first channel assembly of parallel channels is aligned with the air input opening of the second channel assembly of parallel channels.

5. The air treatment apparatus of claim 4, wherein a one-way valve is situated between the air output opening of the first channel assembly of parallel channels and the air input opening of the second channel assembly of parallel channels to couple the first channel assembly to the second channel assembly in a serial manner.

6. The air treatment apparatus of claim 1, wherein the air output of the first channel assembly of parallel channels is coupled to the main air output.

7. The air treatment apparatus of claim 5, wherein the at least one air mover is an impeller that is situated between the output of the first layer of channels and the main air output to pull air through the PTC for treatment.

8. The air treatment apparatus of claim 1, wherein the at least one filter includes a first filter situated in the air flow path downstream of the PTC.

9. The air treatment apparatus of claim 8, further comprising a first funnel situated between the first filter and the PTC.

10. The air treatment apparatus of claim 8, wherein the at least one filter further includes a second filter situated downstream of the PTC.

11. The air treatment apparatus of claim 8, further comprising a second funnel situated between the PTC and the first filter.

12. The air treatment apparatus of claim 2, wherein the first and second channel assemblies of parallel channels are configured as a vertically stacked array of channel assemblies.

13. The air treatment apparatus of claim 3, further comprising a first pipe that couples the air output opening of the first channel assembly of parallel channels to the air input opening of the second channel assembly of parallel channels.

14. The air treatment apparatus of claim 13, wherein the first channel assembly of parallel channels is vertically stacked with respect to the second channel assembly of parallel channels.

15. The air treatment apparatus of claim 13, wherein the first channel assembly of parallel channels is horizontally stacked with respect to the second channel assembly of parallel channels.

16. The air treatment apparatus of claim 15, further comprising a one-way valve connecting an air output of the first channel assembly to an air input of the second channel assembly.

17. The air treatment apparatus of claim 13, wherein
the at least one filter includes a first filter situated upstream of the PTC, the at least one filter includes a second filter situated downstream of the PTC, the air treatment apparatus further comprising:
a first funnel situated between the first filter and an upstream side of the PTC;
a second funnel situated between the second filter and the downstream side of the PTC;.
a second pipe coupling an output of the first funnel to the air input opening of the PTC, and
a third pipe coupling the air output opening of the PTC to an input of the second funnel.

18. The air treatment apparatus of claim 3, further comprising an interposer assembly including first and second interposer components that mate with one another, the first interposer component being situated at a bottom surface of the first channel assembly, the second interposer component being situated at a top surface the second channel assembly.
